(19) United States
(12) Patent Application Publication
(10) Pub. No.: US 2025/0034554 A1
(43) Pub. Date: Jan. 30, 2025

(54) METHODS AND COMPOSITIONS FOR PREPARING AN ARRAYED LIBRARY OF CELLS

(71) Applicant: The Regents of the University of California, Oakland, CA (US)

(72) Inventors: Amy Yi-Tyng Chang, Oakland, CA (US); Eric D. Chow, Oakland, CA (US); Brandon Kwan-Leong, Oakland, CA (US)

(21) Appl. No.: 18/782,679

(22) Filed: Jul. 24, 2024

Related U.S. Application Data
(60) Provisional application No. 63/516,276, filed on Jul. 28, 2023.

(51) Int. Cl. C12N 15/10; C12M 1/12; C12M 1/30; C12M 1/36; C40B 60/00; G01N 35/00; G01N 35/10

(52) U.S. Cl. CPC C12N 15/1065; C12M 25/04; C12M 33/02; C12M 41/48; C12N 15/1058; C40B 60/00; G01N 35/0099; G01N 35/10

(57) ABSTRACT

Provided are methods and compositions for creating an arrayed library of cells. In particular, the methods and compositions provided herein improve the selection and transfer of cells by reducing contamination, increasing throughput, and improving consistency of transfer. Such methods and compositions include automated colony selection employing a solid barrier mounted on an automated apparatus for automatic handling of one or more pick tools. The solid barrier is configured to prevent undesired transfer of cells from a pick tool into the destination receptacle. Such methods and compositions also include acoustic transfer of cells in a high-density media by an acoustic liquid handler. The methods and compositions further include pooling the cells using a bitcode sample pooling scheme for high-throughput sequencing and deconvoluting the obtained nucleic acid sequences to identify the cells from which the sequences originated.

### CROSS-REFERENCE

This application claims the benefit of U.S. Provisional Patent Application No. 63/516,276 filed Jul. 28, 2023, which application is incorporated herein by reference in its entirety.

### I. INTRODUCTION

There is a need for compositions and methods that provide robust, consistent cell transfer, e.g., when generating arrayed libraries of cells using automated colony pickers and liquid handlers. Such is provided herein.

### II. SUMMARY

The work described in the experimental examples below led to the surprising finding by the inventors that an unexpected large amount of cross-contamination can occur (e.g., between wells) when using an automated pick tool to pick colonies into a destination receptacle. To solve this problem, a solid barrier (also referred to herein as a shield) was designed and produced to prevent transfer of cells from inactive pick tools to undesired locations of the destination receptacle.

The Inventors also surprisingly discovered that poor and inconsistent acoustic transfer of cells can result when cells are cultured and incubated in standard media such as LB growth media. They determined this was due to the fact that during acoustic transfer, the droplets that are ejected (via the application of acoustic energy) form at the meniscus/surface of the fluid, and cells were becoming concentrated, e.g., sometimes in dense, circular pellets, at the bottom of the receptacle. Improved, consistent acoustic transfer of cells was then achieved using high-density growth media, which provided increased buoyancy, thus preventing cells from concentrating at the bottom. The high-density growth media also improved (i.e., reduced) cross-contamination.

Provided are methods and compositions for creating an arrayed library of cells (e.g., microorganisms). Such methods include automated colony selection employing a solid barrier mounted on an automated apparatus for automatic handling of one or more pick tools. The automated apparatus selects colonies of cells from a colony source and deposits each of the colonies into a unique location of a destination receptacle (a “first” receptacle). The solid barrier is configured to prevent transfer of cells from an inactive pick tool into an undesired location of the destination receptacle, and thus is positioned between the destination receptacle and the inactive pick tools of the automated apparatus. Such methods also include acoustic transfer of cells in a high-density media by an acoustic liquid handler. The cells are acoustically transferred from each said unique location of the destination receptacle to a “second” receptacle (a pooling receptacle) to produce pools of picked cells. Nucleic acids of the pooled cells are then sequenced, and the obtained sequences are deconvoluted to assign a nucleic acid genotype to each unique location of the first receptacle.

In some cases, the automated apparatus for automated handling of one or more pick tools is a Hudson Robotic RapidPick MP. In some cases, the acoustic liquid handler is an Echo 525 Liquid Handler. In some embodiments, the cells are genetically modified. In some cases, the cells are genetically modified to include a member of a molecular library (e.g., a guide RNA library, a transgene library, an shRNA library, a long noncoding RNA library, an open reading frame expression library, a library of mutated nucleic acid sequences, a library of viral sequences, and the like). In some cases, the molecular library includes nucleic acid barcode sequences (i.e., the members of the molecular library are barcoded). In some cases, the molecular library comprises vectors and each of the vectors comprises one or more guide RNAs. In some embodiments, the genetically modified cells are cultivated to form cell colonies, and each cell colony includes the same member of the molecular library.

In some embodiments, the cells comprise a genetic modification introduced by transformation, transduction, or transfection. In some cases, the cells (e.g., microorganisms) are virally transduced. In some cases, the cells are bacteria or fungi. In some embodiments, a subject method includes a step of producing the colonies of cells (e.g., microorganisms).

In some embodiments, adjustments or modifications can be made to the automated apparatus for automatic handling of one or more pick tools to reduce the speed at which the active pick tool is extended and retracted during the process of colony selection.

In some embodiments, the solid barrier may include any suitable material, such as, for example, acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.

In some embodiments, the automated colony selection destination receptacle (i.e., the “first” receptacle), may be any multi-well plate suitable for use with an acoustic liquid handler. As described above, the automated colony selection destination receptacle (i.e., the “first” receptacle) can be the source from which microorganisms are acoustically transferred in a high-density media to a pooled receptacle (i.e., the “second” receptacle). In some cases, the high-density media includes LB broth and between 1-10% glycerol. In some cases, the high-density media includes 2.5% glycerol. In some cases, the high-density media is added to the automated colony selection destination receptacle (i.e., the “first” receptacle) prior to performing automated colony selection.

In some embodiments, the one or more locations of the pooled receptacle (i.e., the “second” receptacle) into which cells are transferred from the automated colony selection destination receptacle (i.e., the “first” receptacle) are determined using a bitcode sample pooling scheme. In some cases, the bitcode sample pooling scheme is a 24-bitcode sample pooling scheme. In some embodiments, the acoustic transfer of cells using an acoustic liquid handler occurs when the cells are in early- to mid-log phase.

In some embodiments, deconvolution of the obtained nucleic acid sequences of the cells comprises using an unsupervised machine learning algorithm. In some cases, the machine learning algorithm is a Gaussian Mixture Model algorithm.

In some embodiments, the nucleic acid genotype and associated unique location of the first receptacle identified through deconvolution are used to produce a curated arrayed library of cells. In some cases, cells are selected and transferred from the automated colony selection destination receptacle (i.e., the “first” receptacle) to a new receptacle (i.e., a “third” receptacle) to produce a curated arrayed library of cells. In some cases, following automated colony selection and prior to pooling the cells, a copy of the automated colony selection destination receptacle (i.e., the “first” receptacle) is prepared to produce a “copy” receptacle. In some cases, cells are selected and transferred from the copy receptacle to a new receptacle (i.e., a “third” receptacle) to produce a curated arrayed library of cells.

Provided also are compositions for selecting and transferring cells (e.g., microorganisms). In some cases, such compositions may also be used for creating arrayed libraries. Such compositions include a solid barrier mounted on an automated apparatus for automatic handling of one or more pick tools. Such compositions also include an automated colony selection destination receptacle (a “first” receptacle) containing a high-density media. The composition further includes an acoustic liquid handler. In some embodiments, the composition further includes cells. (e.g., cells genetically modified to include a member of a molecular library). In some cases, the high-density media in the automated colony selection destination receptacle (i.e., the “first” receptacle), includes LB broth and between 1-10% glycerol. In some cases, the high-density media in the automated colony selection destination receptacle (i.e., the “first” receptacle) includes 2.5% glycerol. In some embodiments, the composition further includes an acoustic transfer target receptacle (i.e., the “second” receptacle). In some embodiments, the composition further includes a computer configured for deconvolution of nucleic acid sequences. In some cases, the computer is configured to perform deconvolution using an unsupervised machine learning algorithm. In some cases, the machine learning algorithm is a Gaussian Mixture Model algorithm.

Provided also are methods for selecting and transferring cells (e.g., microorganisms). Such methods include automated colony selection employing a solid barrier mounted on an automated apparatus for automatic handling of one or more pick tools. The automated apparatus selects colonies of cells from a colony source and deposits each of the colonies into one or more locations of a destination receptacle (a “first” receptacle). Such methods also include acoustic transfer of cells in a high-density media by an acoustic liquid handler. The cells are acoustically transferred from the one or more locations of the destination receptacle to one or more locations of a “second” receptacle (a pooling receptacle).

Provided also are methods and compositions for acoustically transferring cells. Such methods and compositions include obtaining a receptacle (i.e., a “first” receptacle) containing one or more cells in a high-density media and using an acoustic liquid handler to acoustically transfer at least one or more of said cells to a target (i.e., a “second” receptacle).

Provided also are methods for reducing contamination during automated colony selection. Such methods include automated colony selection employing a solid barrier mounted on an automated apparatus for automatic handling of one or more pick tools.

Provided also is a solid barrier mounted on an automated apparatus for automatic handling of one or more pick tools.

Reagents, compositions, and kits/systems that find use in practicing the subject methods are provided.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A-1B depict an example solid barrier (shield) mounted to an automated colony picker (automated apparatus for automatic handling of one or more pick tools).

FIG. 2 depicts a schematic drawing of a first bracket of an example solid barrier (shield).

FIG. 3 depicts a schematic drawing of a second bracket of an example solid barrier (shield).

FIG. 4 depicts a schematic drawing of a bottom plane of an example solid barrier (shield).

FIG. 5 depicts a schematic drawing of a side plane of an example solid barrier (shield).

FIG. 6 depicts a schematic drawing of a top (that includes an upper plane and a lower plane) of an example solid barrier (shield).

FIG. 7A-7B FIG. 7A (top) depicts a sample image of acoustically transferred cells from different source media. FIG. 7A (bottom) depicts sample images of destination plates with E. coli incubated in LB (left, pellets observed), or in high-density media (right, pellets not observed). FIG. 7B depicts results from molecular library identification before (‘first’ experiment) and after (‘second’ experiment) incorporating protocol improvements described herein—plotted relative to simulations of the theoretical prediction(identification) rate as a function of library skew and pick-coverage (dotted curve).

FIG. 8 depicts a cell growth curve to identify time points for early-to-mid log phase.

FIG. 9 depicts an example schematic workflow from a pooled molecular library, to colony formation, to colony picking, to bitcode pooling, to sequencing and nucleic acid genotype determination.

FIG. 10 depicts an example schematic computational workflow.

FIG. 11 depicts results from example experiments demonstrating machine learning thresholding to distinguish spurious from true reads and to binarize read count data.

### IV. DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Compositions and Methods

As noted above, provided are methods and compositions (e.g., for creating an arrayed library of cells such as microorganisms) that include an automated apparatus for automatic handling of one or more pick tools for performing automated selection of cells into one or more locations of a destination receptacle (a “first” receptacle). The automated apparatus includes a mounted solid barrier to prevent transfer of cells from an inactive pick tool into an undesired location of the first receptacle.

### Cells

Provided herein are methods and composition for the selection and/or transfer of cells. Methods and compositions of the present disclosure may be applied to any type of biological organism that can be cloned out of a pool including, but not limited to, bacteria, yeast, and mammalian cells.

In some embodiments, the subject cells are microorganisms (i.e., single cell microorganisms). A microorganism can include one or more of the following features: aerobe, anaerobe, filamentous, non-filamentous, monoploid, diploid, auxotrophic and/or non-auxotrophic. In certain embodiments, a microorganism is a prokaryotic microorganism (e.g., bacterium), and in certain embodiments, a microorganism is a non-prokaryotic microorganism. In some embodiments, a microorganism is a eukaryotic microorganism (e.g., yeast, fungi, amoeba).

Examples of microorganisms suitable for the methods and compositions of the present disclosure include, but are not limited to, Streptomyces bacteria (e.g., Streptomyces rubiginosus, Streptomyces murinus), Escherichia bacteria (e.g., E. coli), Bacillus bacteria (e.g., B. subtilis. B. megaterium, B. stearothermophilus), Saccharomyces yeast (e.g., S. cerevisiae, S. bayanus, S. pastorianus, S. carlsbergensis), Aspergillus fungi (e.g., A. parasiticus, A. nidulans), Lactobacillus bacterial (e.g., Lactobacillus pentosus), Cyanobacteria, and the like.

In some embodiments, the subject cells are tissue cells or cell lines. The subject cells may be primary cells that are directly isolated from a tissue sample, (e.g., fibroblasts obtained from skin biopsies and hepatocytes isolated from liver explants), transformed cells that are immortalized either naturally or through genetic manipulation (e.g., Chinese hamster ovary (CHO), HeLa, human umbilical vein endothelial cells (HUVEC)), or self-renewing cells that carry the capacity to differentiate into a diversity of other cells types (e.g., embryonic stem cells, induced pluripotent stem cells, neural and intestinal stem cells).

Tissue cells or cell lines may be obtained from any type of organism, including plants and animals. For example, tissue cells or cell lines may be obtained from mammals (e.g., rodents (rats, mice, hamsters, guinea pigs), non-human primates, humans, canines, felines, ungulates (e.g., equines, bovines, ovines, porcines, caprines), lagomorphs), invertebrates (e.g., a cnidarian, an echinoderm, a worm, a fly, etc.), non-mammalian vertebrates (e.g., a fish (e.g., zebrafish, puffer fish, gold fish, etc.)), amphibians (e.g., salamander, frog, etc.) reptiles, or birds. Tissue cells or cell lines may be derived from any type of tissue, including, but not limited to, brain tissue, prostate tissue, urinary tract tissue, gall bladder tissue, tissues from the uterus and other portions of the female reproductive tract, vasculature tissue, tissues from the intestines and other portions of the lower alimentary tract, tissues from the stomach and other portions of the upper alimentary tract, tissues from the liver and other digestive organs, lung tissue, skin, mucus membranes, kidney tissue, reproductive organs tissue, joints, other organs or soft tissues of the body, root tissue, seed tissue, meristem tissue, callus tissue, bud tissue, and the like. Various cell lines are also commercially available and include, but are not limited to, Chinese hamster ovary (CHO), COS-7, Vero, MDCK, Sf9, HeLa, SH-SY5Y, HEK-293, MCF-7, H1, H9, and the like.

Cells of the present disclosure may be provided in any suitable form. For example, such cells may be provided in liquid culture or solid culture (e.g., agar-based medium), which may be a primary culture or may have been passaged (e.g., diluted and cultured) one or more times. In some cases, cells may be adherent and grow while adhered to a culture surface. Adherent cells may be provided adhered to a culture surface or detached from the culture surface. In other cases, cells are non-adherent and grow suspended in a growth medium. Non-adherent cells may be provided as cells in suspension (e.g., eukaryotic cells in suspension, mammalian cells in suspension, and the like). Methods of culturing cells are well known in the art. Cells also may be provided in frozen form or dry form (e.g., lyophilized). Cells may be provided at any suitable concentration. In some embodiments, the cells are provided in a form that is suitable for use with automated colony selection instruments and/or acoustic liquid handlers.

In some cases, the cells may be cultivated to form colonies. As used herein, “cell colonies” are clusters of genetically identical cells originating from a single cell. Cell colonies may comprise any of the cell types described above (e.g., single cell microorganisms (e.g., bacteria, yeast, parasites, fungi), tissue cells, or cells from cell lines). When cultured under the appropriate conditions, a single cell will divide into two daughter cells, which are identical to the original cell. The cells will continue to divide to form a cluster of identical cells (clones). Each cluster of identical cells is a colony, and all cells within the colony are genetically identical to the original cell. Where the original cell contains a genetic modification, each of the clones will also have the same genetic modification.

Methods of cell culture for producing colonies are known in the art and any convenient method can be used. Receptacles and growth media suitable for producing colonies are known to one of ordinary skill in the art and any convenient receptacle/media (e.g., culture dish with LB/agar) can be used. In some cases, the cell colonies are produced using receptacles/media suitable for use with an automated colony picker. Examples of growth media suitable for include, but are not limited to, agar, blood agar, nutrient agar, McConkey agar, chocolate agar, and the like.

Cell colonies can be counted and/or selected and transferred to another location. Target cell colonies can be discriminated on the basis of a variety of attributes, such as shape, size, color or molecular content that may be within the cell, in the membrane or secreted, or by any combination of multiple attributes. Some types of cell colonies are visible to the eye. Additionally, many growth media used for culturing include indicator compounds to indicate the presence of a cell colonies. Indicator compounds include, for example, pH indicators, chromogenic enzyme substrates, and redox indicators. The indicator compounds, when converted directly or indirectly to a product, typically impart a color change to the cell colony and/or the growth medium surrounding the colony. The color change often makes it easier to detect the presence of the cell colony in the growth medium (e.g., it improves the color contrast between the colony and the growth medium) and may the color change also may serve to differentiate a particular colony that reacts with a particular indicator compound from another cell colony that does not react with that indicator compound.

### Genetic Modification of Cells

A cell of the present disclosure is often suitable for genetic manipulation. As used herein, a “genetically modified cell” is a cell which has been modified by any suitable nucleic acid addition, removal, or alteration.

Genetic modifications include, without limitation, insertion of one or more nucleotides in a native nucleic acid of a cell in one or more locations, deletion of one or more nucleotides in a native nucleic acid of a cell in one or more locations, modification or substitution of one or more nucleotides in a native nucleic acid of a cell in one or more locations, insertion of a non-native nucleic acid into a cell (e.g., insertion of an autonomously replicating vector), and removal of a non-native nucleic acid in a host organism (e.g., removal of a vector).

Examples of methods useful for generating a genetically modified cell include, but are not limited to, introducing a heterologous polynucleotide (e.g., nucleic acid or gene integration, also referred to as “knock in”), removing an endogenous polynucleotide, altering the sequence of an existing endogenous nucleic acid sequence (e.g., site-directed mutagenesis), disruption of an existing endogenous nucleic acid sequence (e.g., knock outs and transposon or insertion element mediated mutagenesis), selection for an altered activity where the selection causes a change in a naturally occurring activity that can be stably inherited (e.g., causes a change in a nucleic acid sequence in the genome of the organism or in an epigenetic nucleic acid that is replicated and passed on to daughter cells), PCR-based mutagenesis, and the like.

A nucleic acid (e.g., also referred to herein as nucleic acid reagent, target nucleic acid, target nucleotide sequence, nucleic acid sequence of interest or nucleic acid region of interest) suitable for use in genetically modifying a cell can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). A nucleic acid can also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

In some cases, a nucleic acid is a plasmid, phage, autonomously replicating sequence (ARS), centromere, artificial chromosome, yeast artificial chromosome (e.g., YAC), vector, cosmid, or other nucleic acid able to replicate or be replicated in a host cell. In certain embodiments a nucleic acid can be from a library or can be obtained from enzymatically digested, sheared, or sonicated genomic DNA (e.g., fragmented) from an organism of interest. In some embodiments, a nucleic acid is introduced to a cell by transformation, transfection, or transduction. In some embodiments, a cell is genetically modified by delivery of a nucleic acid via viral transduction. Methods of transformation, transfection, and transduction are known in the art and any convenient method can be used.

A molecular library (e.g., nucleic acid library or collection of nucleic acid sequences) can include one or more “members” of the library, wherein each member is a different nucleic acid sequence of interest. In some embodiments, a library may be designed and assembled to be representative of a plurality of predetermined nucleic acid or polypeptide sequences that are selected or provided (e.g., provided by a customer). Examples of nucleic acid sequences that may be contained in the library include, but are not limited to, a plurality of guide RNAs, such as CRISPR/Cas guide RNAs, a plurality of transgenes, a plurality of shRNAs, a plurality of siRNAs, a plurality of miRNAs, a plurality of long noncoding RNAs (lncRNAs), a plurality of nucleic acids comprising open reading frames (ORFs), a plurality of mutated nucleic acid sequences, and a plurality of viral nucleic acid sequences.

As used herein, a “molecular library” includes at least 2 members (e.g., at least 5, at least 10, at least 100, at least 1000, etc.). In some cases, a subject molecular library includes at least 50 members (e.g., at least 100, at least 1000, at least 5000, at least 10000). In some cases, a subject library includes at least 100 members (e.g., at least 500, at least 1000, at least 5000, at least 10000). In some cases, a subject library includes at least 500 members (e.g., at least 1000, at least 5000, at least 10000). In some cases, the number of members in a subject library is in a range of from 2-30000 members (e.g., from 2-100, from 100-500, from 500-1000, from 1000-5000, from 5000-10000, from 10000-20000). In some cases, the number of members in a subject library is in a range of from 50-200 members (e.g., from 50-100, from 100-200). In some cases, the number of members in a subject library is in a range of from 500-1000 members (e.g., from 500-700, from 700-9000, from 900-1000). In some cases, the number of members in a subject library is in a range of from 1000-5000 members (e.g., from 1000-2000, from 2000-3000, from 3000-5000). In some cases, the number of members in a subject library is in a range of from 10000-30000 members (e.g., from 10000-15000, from 15000-25000, from 25000-30000).

In some embodiments, a library of nucleic acid sequences may comprise constructs assembled from oligonucleotides or nucleic acid fragments. In some embodiments, a library contains a plurality of related nucleic acids that include predetermined sequence differences at only a subset of positions. In some cases, the construct is a vector. In some cases, a construct includes two or more nucleic acid sequences of interest (e.g., a vector including two guide RNAs of interest).

Multiple barcoded constructs can be assembled as described herein. In some embodiments, a barcode library can be used to assemble and barcode multiple constructs, such that each nucleic acid construct can be tagged with a unique barcode. In other embodiments, multiple constructs may be assembled from multiple internal target sequence fragments and unique barcode sequences.

In some embodiments, the barcoded nucleic acids of a molecular library of nucleic acids are introduced into a plurality of cells. In some cases, the cells are further inoculated into a culture dish to form colonies. In some cases, each originating cell of a colony contains one member of the molecular library and each cell in a colony therefore contains the same member of the molecular library.

### Automated Colony Selection

In some embodiments of the present disclosure, colonies of cells are selected from a colony source and deposited in one or more locations of a destination receptacle (i.e., a colony selection process). In some cases, colonies are selected and deposited manually by an experimenter. In some cases, colonies are selected and deposited using an automated colony picker (i.e., an automated colony selection process).

### Solid Barrier

Provided herein are methods and compositions for automated colony selection that utilize a solid barrier (a shield) to reduce cross-contamination during automated selection and transfer of cell colonies. Cross-contamination occurs when cells are transferred into an undesired location of a destination receptacle. As such, to prevent the undesired transfer of cells (e.g., from an inactive pick tool into an undesired location of a destination receptacle), a subject solid barrier is arranged such that it forms a physical barrier across which cells cannot pass. The solid barrier is positioned between the destination receptacle and one or more inactive pick tools to prevent the transfer of cells from the inactive pick tools to undesired locations of the destination receptacle. As such, the solid barrier can be a plane (also referred to herein as a bottom plane) positioned above and parallel to the destination receptacle, but below the inactive pick tools of the automated colony picker.

The solid barrier includes an opening (a port) to allow an active pick tool to penetrate past the solid barrier to access the destination receptacle. The port can be any convenient size and shape (e.g., a circular hole, a square hole, a notch, etc.). Active and inactive pick tools are described elsewhere herein. In some cases, the port is a circle with a diameter in a range of from 3-20 mm (e.g., 3-18 mm, 3-15 mm, 3-12 mm, 3-10 mm, 3-8 mm, 3-5 mm, 5-20 mm, 5-18 mm, 5-15 mm, 5-12 mm, 5-10 mm, 5-8 mm, 8-20 mm, 8-18 mm, 8-15 mm, 8-12 mm, 8-10 mm, 10-20 mm, 10-18 mm, 10-15 mm, or 10-12 mm).

In some cases, the solid barrier is mounted to (in some cases fastened/attached to) the automated colony picker. In some such cases, the solid barrier hangs from or clamps onto the automated colony picker. In some cases, the solid barrier is attached/fastened to the automated colony picker (e.g., using any convenient fastener, e.g., hook and loop, stick tape, screws such as machine screws, and the like). The term “mounted” is intended herein to include scenarios in which the solid barrier is manufactured as a part (e.g., a seamless part) of the automated colony picker—thus, an automated colony picker can be manufactured to include a subject solid barrier (a plane that is above and parallel to the destination receptacle, but below the inactive pick tools).

In some cases, the solid barrier (shield) includes pieces/parts in addition to the barrier that is above and parallel to the destination receptacle. These parts can provide additional shielding in different planes, and/or can provide for mounting and/or fastening of the shield to the automated colony picker. For example, in some cases, a subject solid barrier includes one or more top planes (e.g., to allow for mounting, e.g., fastening, to the automated colony picker). In some cases, a subject solid barrier includes one or more brackets, which can be used, e.g., for mounting and in some cases fastening the solid barrier to the automated colony picker. In some cases, such bracket(s) can be used as an assembly point, e.g., to attached the bottom plane to a top plane and/or to a side plane. In some cases, a subject solid barrier includes a side plane (e.g., for provide additional protection against cross contamination).

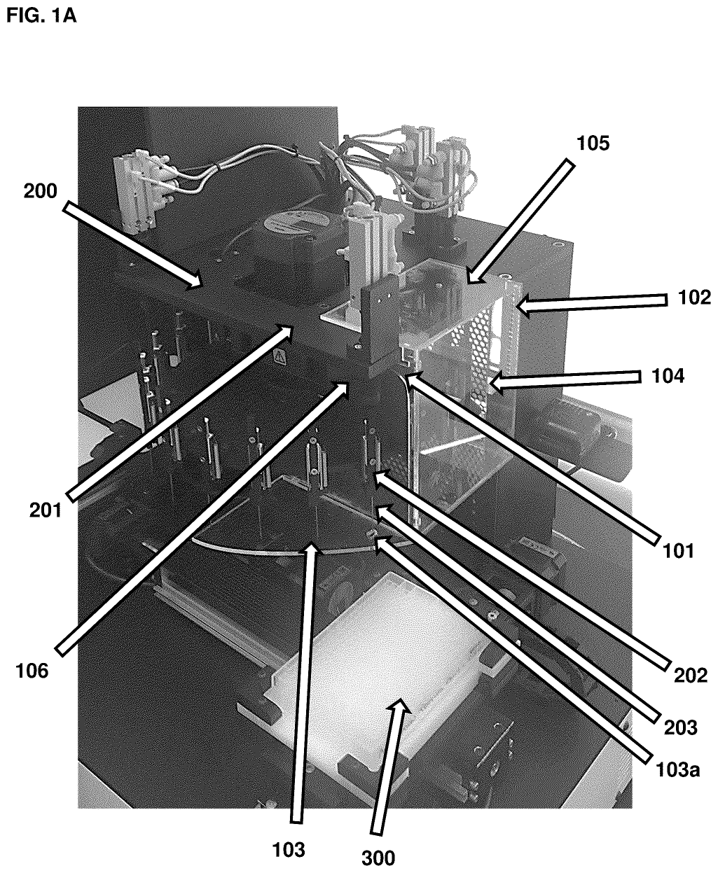

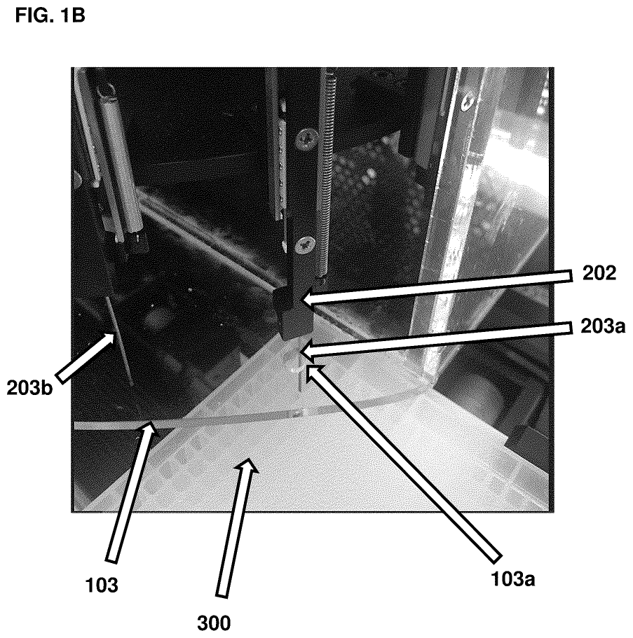

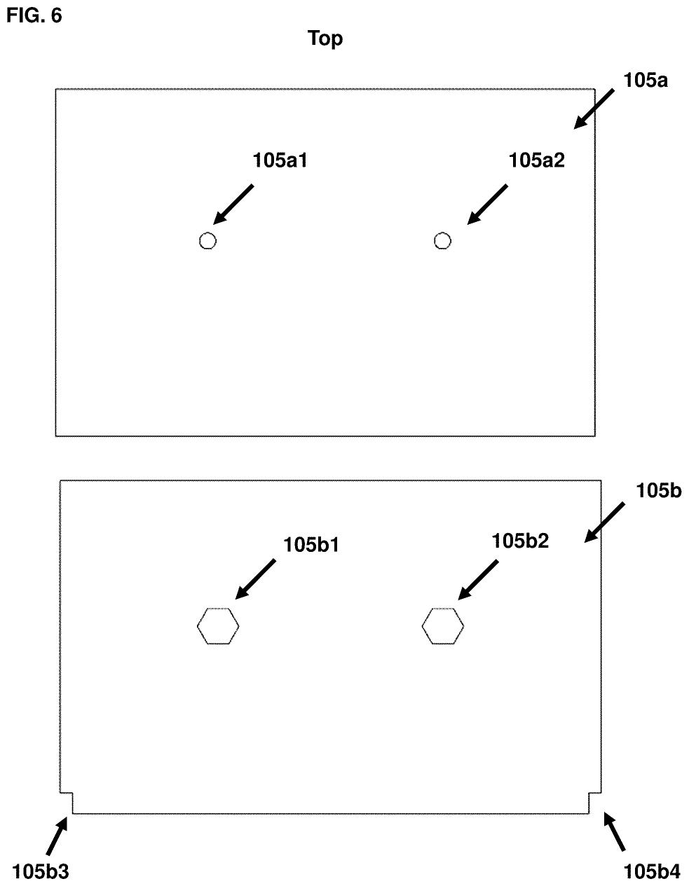

An example solid barrier that includes various additional parts (in addition to the bottom plane) is depicted in FIG. 1A-B through FIG. 6. The shield 100 depicted in FIGS. 1A-1B and FIG. 2-FIG. 6 includes multiple parts (101-106). The shield 100 creates a physical barrier between the picking tool 203 and the destination receptacle 300 (e.g., well in a 384-well plate) where/when the destination receptacle 300 is not actively being inoculated. The bottom plane 103 of the shield incorporates a port 103a through which an active picking tool 203a will pass when selected to perform an inoculation. Contamination is minimized when an inactive picking tool 203b moves over the destination receptacle 300 because the bottom of the shield 103 is a physical barrier between the two. The parts of the shield 100 of the example embodiment depicted in the figures are made of acrylic and joined by glue, and are as follows.

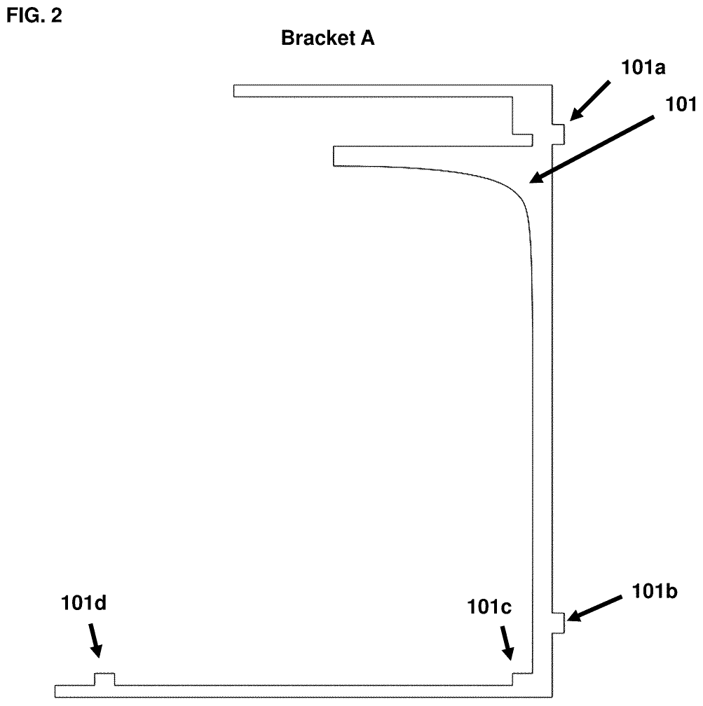

Bracket A 101 hangs from the roof of the colony picker 201. The picking tools 203 (which rotate in a counterclockwise manner about the center of the colony picker 200) approach Bracket A 101 as they approach the destination receptacle 300. Along with Bracket B 102, it forms the connection point for all other parts. See FIG. 1A and FIG. 2. Tabs 101a-101d are used in this example embodiment to aid in fitting together the pieces, e.g., tabs 101a/b are used for fitting Bracket A 101 with the notches of side plane 104, and tabs 101c/d are used for fitting Bracket A with the notches of bottom plane 103.

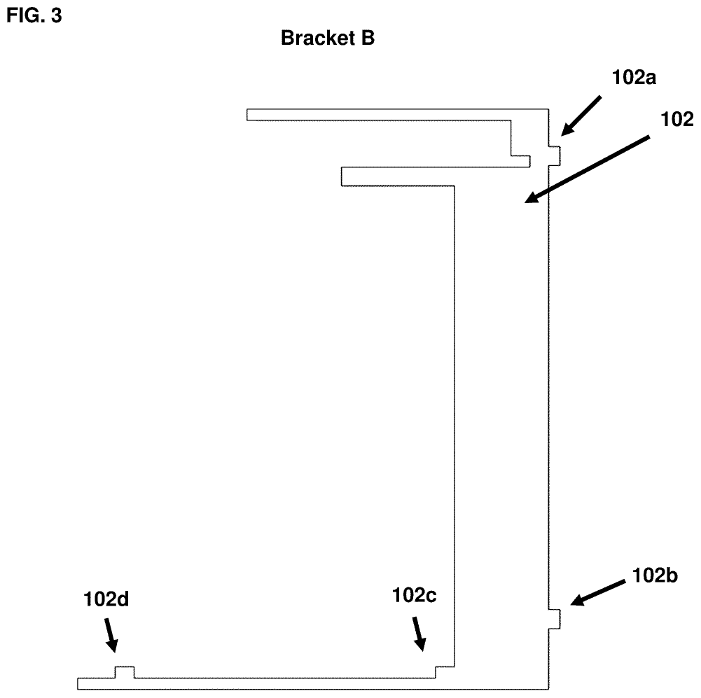

Bracket B 102 hangs from the roof of the colony picker 201. The picking tools 203 egress away from Bracket B 102 as they egress away from the destination receptacle 300. Along with Bracket A 101, it forms the connection point for all other parts. See FIG. 1A and FIG. 3. Tabs 102a-102d are used in this example embodiment to aid in fitting together the pieces, e.g., tabs 102a/b are used for fitting Bracket B with the notches of side plane 104, and tabs 102c/d are used for fitting Bracket B 102 with the notches of bottom plane 103.

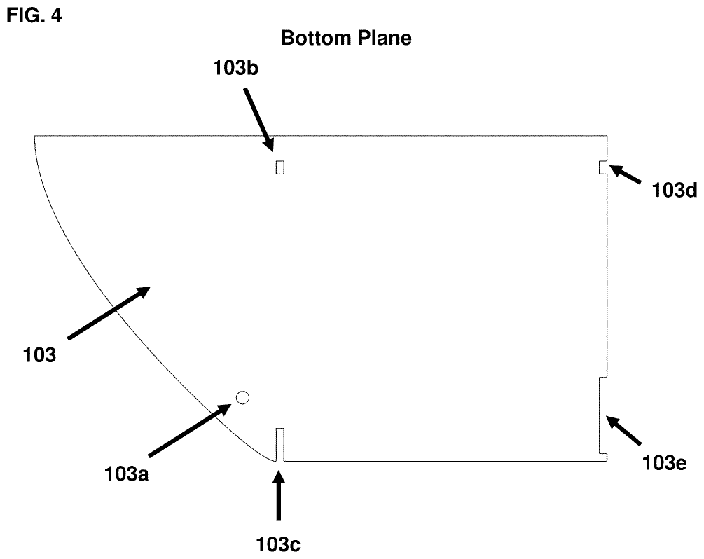

Bottom plane 103 is above and parallel to the top of the destination plate 300 and is below the inactive pick tools 203, allowing the destination receptacle 300 to move freely underneath. A port 103a allows the active picking tool to pass through to perform an inoculation. See FIGS. 1A-1B and FIG. 4. Notches/holes 103a-103d are used in this example embodiment to aid in fitting together the pieces, e.g., notches/holes 103b/c are used for fitting bottom plane 103 with the tabs of bracket A 101, and notches 103d/e are used for fitting bottom plane 103 with the tabs of bracket B 102.

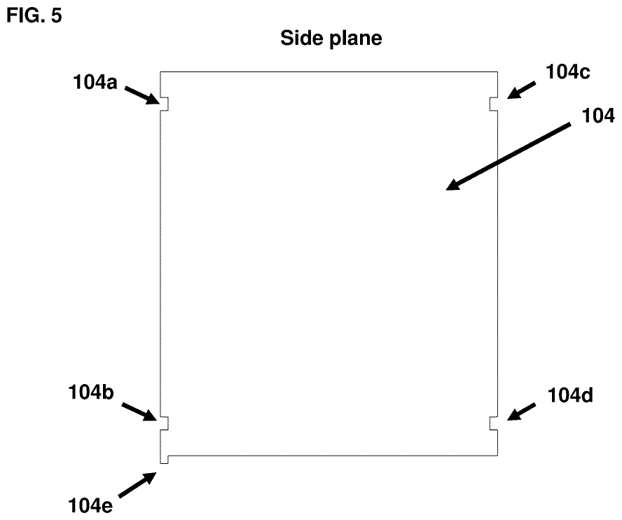

Side plane 104 is perpendicular to the top of the destination receptacle 300, providing a physical barrier between the picking tools 203 and destination receptacle 300 when the destination receptacle 300 is not completely covered by the bottom plane 103. See FIG. 1A and FIG. 5. Notches 104a-104d are used in this example embodiment to aid in fitting together the pieces, e.g., notches 104a/b are used for fitting side plane 104 with the tabs of bracket A 101a/b, and notches 104c/d are used for fitting side plane 104 with the tabs of bracket B 102a/b. Likewise, tab 104e of is used for fitting side plane 104 with the notch of bottom plane 103c.

The top 105 of the shield is made of two planes, an upper top plane 105a and a lower top plane 105b, directly resting above and below the roof of the colony picker 201, respectively. The upper and lower top planes 105a/b are fastened by machine screws, providing the anchor point to the colony picker. See FIG. 1A and FIG. 6. Openings 105a1/a2 and 105b1/b2 facilitate fastening. Notches 105b3 and 105b4 are used in this example embodiment to aid in fitting together the pieces.

A subject solid barrier can be any convenient shape and size, and in some cases is shaped to mimic the contours of the automated colony picker. In some embodiments, a subject solid barrier is large enough to cover (i.e., is positioned over) the entirety of the destination receptacle (e.g., can cover and protect all wells of a multi-well destination plate). In some embodiments, a subject solid barrier covers a portion of the destination receptacle (e.g., some but not all of the wells of a multi-well destination plate). In some cases, a subject solid barrier covers 30% or more of the destination receptacle (e.g., 40% or more, 50% or more, 60% or more, 70% or more, 80% or more, 90% or more, 95% or more, or 100%). In some cases, a subject solid barrier covers 50% or more of the destination receptacle (e.g., 60% or more, 70% or more, 80% or more, 90% or more, 95% or more, or 100%). In some cases, a subject solid barrier covers 50% or more of the destination receptacle (e.g., 60% or more, 70% or more, 80% or more, 90% or more, 95% or more, or 100%).

The parts of the solid barrier (e.g., bottom plane as well as top plane(s), bracket(s), side plane) can be any convenient length. In some embodiments, the bottom plane (or any part of the solid barrier) has a length in a range of from 50-650 mm (e.g., 50-600 mm, 50-550 mm, 50-500 mm, 50-450 mm, 50-400 mm, 50-350 mm, 50-300 mm, 50-250 mm, 50-200 mm, 50-150 mm, 50-100 mm, 100-650 mm, 100-600 mm, 100-550 mm, 100-500 mm, 100-450 mm, 100-400 mm, 100-350 mm, 100-300 mm, 100-250 mm, 100-200 mm, 100-150 mm, 150-650 mm, 150-600 mm, 150-550 mm, 150-500 mm, 150-450 mm, 150-400 mm, 150-350 mm, 150-300 mm, 150-250 mm, 150-200 mm, 200-650 mm, 200-600 mm, 200-550 mm, 200-500 mm, 200-450 mm, 200-400 mm, 200-350 mm, 200-300 mm, 200-250 mm, 250-650 mm, 250-600 mm, 250-550 mm, 250-500 mm, 250-450 mm, 250-400 mm, 250-350 mm, or 250-300 mm). The parts of the solid barrier (e.g., bottom plane as well as top plane(s), bracket(s), side plane) can be any convenient width. In some embodiments, the bottom plane (or any part of the solid barrier) has a width in a range of from 50-650 mm (e.g., 50-600 mm, 50-550 mm, 50-500 mm, 50-450 mm, 50-400 mm, 50-350 mm, 50-300 mm, 50-250 mm, 50-200 mm, 50-150 mm, 50-100 mm, 100-650 mm, 100-600 mm, 100-550 mm, 100-500 mm, 100-450 mm, 100-400 mm, 100-350 mm, 100-300 mm, 100-250 mm, 100-200 mm, 100-150 mm, 150-650 mm, 150-600 mm, 150-550 mm, 150-500 mm, 150-450 mm, 150-400 mm, 150-350 mm, 150-300 mm, 150-250 mm, 150-200 mm, 200-650 mm, 200-600 mm, 200-550 mm, 200-500 mm, 200-450 mm, 200-400 mm, 200-350 mm, 200-300 mm, 200-250 mm, 250-650 mm, 250-600 mm, 250-550 mm, 250-500 mm, 250-450 mm, 250-400 mm, 250-350 mm, or 250-300 mm). The parts of the solid barrier (e.g., bottom plane as well as top plane(s), bracket(s), side plane) can be any convenient thickness, which will likely depend on the type of material used. For example, in some embodiments the bottom plane (or any parts of the solid barrier) has a thickness is a range of from 1-30 mm (e.g., 1-25 mm, 1-20 mm, 1-15 mm, 1-10 mm, 1-5 mm, 1-3 mm, 3-30 mm, 3-25 mm, 3-20 mm, 3-15 mm, 3-10 mm, 3-5 mm, 5-30 mm, 5-25 mm, 5-20 mm, 5-15 mm, 5-10 mm, 10-30 mm, 10-25 mm, 10-20 mm, or 10-15 mm).

A subject solid barrier may be made of any material that is suitable for preventing the transfer of cells from an inactive pick tool to an undesired location of a destination receptacle. Examples of such materials include, but are not limited to, plastics (e.g., acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass (e.g., annealed glass, tempered glass, laminated glass), ceramics, metals (e.g., aluminum, steel), and the like.

### Colony Selection

An automated colony picker includes an automated apparatus that automatically handles one or more pick tools and provides an automated system for locating and collecting a colony of cells from a colony source and transferring said colony to one or more locations of a destination receptacle, i.e., inoculation of the cells in the destination receptacle. The automated apparatus of the automated colony picker conveys a pick tool to the colony source, contacts the pick tool with the desired colony of cells, removes at least a portion of a colony of cells from the surface of the colony source, and deposits the removed cells in one or more locations of destination receptacle. Exemplary automated colony pickers include, but are not limited to, RapidPick MP (Hudson Robotics), RapidPick SP (Hudson Robotics), QPixXE (Molecular Devices), QpixHT (Molecular Devices), Qpix 420 (Molecular Devices), Qpix 450 (Molecular Devices), Qpix 460 (Molecular Devices), ClonePix (Molecular Devices), PIXL (Singer Instruments), Pm-1 (Microtec), Pm-2 (Microtec), EasyPick (Hamilton), and Cavro Omni Flex (Tecan).

In some automated colony pickers, the one or more pick tools are configured as pins. In some automated colony pickers, the one or more pick tools are mounted on a rotating carousel. In some automated colony pickers, the colony source, the destination plate, or both can be moved by the automated colony picker.

In some automated colony pickers, automated image analysis algorithms identify the colonies and provide data for the automated apparatus of the automated colony picker to position the colony source and/or the pick tools for colonies to be picked by the pick tools. In other automated colony pickers, the locations of selected colonies are provided by the user. Using the provided data, the automated apparatus positions a clean pick tool over the selected colony. The pick tool is lowered to contact and remove the desired colony of cells from the surface of the colony source. The automated apparatus then lifts the pick tool and moves the pick tool and/or the destination receptacle such that the pick tool carrying the colony is positioned over a receiving location in the destination receptacle. The pick tool is lowered to deposit the colony, or a portion of the cells in the colony, in the receiving location of the destination receptacle. After depositing the cells, the pick tool is then lifted again. Where it is desired for the cells of the colony to be deposited in more than one location of the destination receptacle, the pick tool and/or the destination receptacle may be repositioned to facilitate deposit of the cells into additional locations of the destination receptacle, and the same pick tool is lowered into the new receiving location of the destination receptacle and lifted after depositing the cells. Where the automated apparatus comprises more than one pick tool, a new, clean pick tool used to select and transfer each separate colony. In some cases, the automated apparatus moves the used pick tool to a cleaning station to clean (e.g., wash, sterilize, etc.) the pick tool prior to reusing the pick tool to select another colony.

An “active” pick tool is considered to be active beginning from the time that the automated apparatus positions the pick tool over the selected colony in the colony source until the pick tool is lifted above the plane of the solid barrier following deposit of the selected colony into the destination receptacle, after which the pick tool is considered inactive. As such, an active pick tool is one that is presently in the process of selecting, removing, and depositing cells from a colony. In contrast, an “inactive” pick tool is any pick tool that is not being used in the process of selecting and collecting a colony or depositing the colony into the destination receptacle. Although the inactive pick tool may be incidentally moved by the automated apparatus in the process of positioning the active pick tool, inactive pick tools are not raised or lowered and do not participate in the selection, removal, or deposit of the colony that is being presently selected and transferred. An inactive pick tool may be contaminated by cells following the use of the pick tool to select a colony, or an inactive pick tool may be clean.

In some automated colony pickers, adjustments to the automated apparatus and pick tools can be made to further refine the automated colony selection process (e.g., selecting and depositing colonies), and in some cases reduce cross-contamination (e.g., via splashing) into neighboring locations of the desired location of the destination receptacle. These adjustments can include, but are not limited to, modifying (e.g., reducing) the speed of pick tool rotation, modifying (e.g., reducing) the pick tool extension/retraction speed (e.g., by reducing air pressure in the pistons driving the pick tools), modifying (e.g., reducing) the liquid dispense rate, and modifying (e.g., increasing) the length of the pick tool (e.g., the depth to which the pick tool will extend into the receptacle). In some cases, an adjustment to an automated colony picker comprises reducing air pressure to the pistons driving the pick tools to reduce the speed of entry and exit of the pick tools when selecting and depositing colonies of cells.

In some cases, the colony source is a receptacle suitable for supporting the growth of cell colonies. In some cases, the colony source is a culture dish. In some cases, the culture dish contains growth media (e.g., with agar) used for growing colonies of cells. Receptacles and growth media suitable for use as a source for an automated colony picker will be known to one of ordinary skill in the art and any convenient receptacle/media (e.g., culture dish with LB/agar) can be used.

In some cases, the destination receptacle is a plate comprising multiple wells (i.e., a multi-well plate). In some embodiments, the destination receptacle may have 6, 12, 24, 48, 96, 384, 1536, 3456, or any number of wells. In some embodiments, the destination receptacle is suitable for use with an acoustic liquid handler. Examples of destination receptacles suitable for use with an acoustic liquid handler (and for use as a destination for an automated colony picker) of the present disclosure include, but are not limited to Echo PP plates and Echo PP Plus plates. In some cases, the destination receptacle is a deep well block.

In some cases, the destination receptacle contains growth media into which the cells of a colony are inoculated. Any growth media suitable to sustain the transferred cells may be used in the destination receptacle. Growth media formulations are known in the art and may include, but are not limited to, ingredients such as antibiotics, hormones, growth factors, amino acids, glucose, vitamins, trace elements, and minerals. Examples of suitable growth media include, but are not limited to, lysogeny broth (LB) (i.e., LB Medium, Luria Broth, Luria-Bertani medium), soybean casein digest broth, tryptic soy broth, phenol red carbohydrate broth, nutrient broth, RPMI-1640, PBS, serum media, Dulbecoo's Modified Eagle Medium (DMEM), and the like.

In some cases, the growth media is a high-density growth media. A high-density growth media is growth media that includes components that increase the density of the growth media relative to standard growth medias, such as lysogeny broth (LB) (i.e., LB Medium, Luria Broth, Luria-Bertani medium). In some cases, a high-density growth media increases the buoyant force on the cells grown in the growth media and thereby reduces the rate of concentration of the cells at the bottom of the receptacle (e.g., pelleting). In some cases, a high-density growth media can include glycerol. In some cases, the high-density growth media includes a range of from 1-10% glycerol (e.g., 1-8%, 1-6%, 1-4%, 1-4%, 2-10%, 2-8%, 2-6%, 2-4%, 2-3%, 3-10%, 3-8%, 3-6%, 4-10%, 4-8%, 4-6%). In some cases, the high-density growth media includes a range of from 1-3% glycerol (e.g., 1%, 1.5%, 2%, 2.5%, or 3%). In some cases, the high-density growth media includes a range of from 3-7% glycerol (e.g., 3-5%, 4-6%, 4-5%). In some cases, the high-density growth media includes 2-3% glycerol. In some cases, the high-density growth media includes 1-3% glycerol. In some cases, the high-density growth media includes about 2.5% glycerol. In some cases, the high-density growth media includes about 5% glycerol. In some cases, a high-density growth media is can be, for example, [LB+2.5% glycerol (LGR), LB+5% glycerol (LGR), LB+2.5% glycerol (derived from Teknova LB+7.5% glycerol), LB+5% glycerol (derived from Teknova LB+7.5% glycerol)]. In some embodiments, the high-density growth media is LB with about 2.5% glycerol. Additional components in growth media formulations are known in the art and may include ingredients such as antibiotics, amino acids, glucose, vitamins, trace elements, and minerals. In some embodiments, the recipe for the high-density growth media is: 15 g tryptone, 7.5 g yeast extract, 7.5 g NaCl, 37.5 mL glycerol, 1462.5 mL water, and 100 g/mL carbenicillinln some cases, a high-density growth media is added prior to performing automated colony selection. In other embodiments, a high-density growth media is added after performing automated colony selection to wells that already contain microorganisms from selected colonies.

### Acoustic Transfer of Cells

Provided are methods and compositions that include acoustic transfer of cells with improved transfer consistency and reduced cross-contamination. In some embodiments, cells are acoustically transferred from the automated colony selection destination receptacle to another receptacle (a target receptacle), e.g., a pooling receptacle. Thus, the automated colony selection ‘destination receptacle’ is also referred to herein as an acoustic transfer ‘source receptacle’. For the sake of clarity, the destination receptacle for the acoustic transfer is referred to herein as a “target receptacle” or simply a “target.”

Liquid transfer machines, which may also be referred to as “liquid handlers”, are used to perform liquid transfers between a source and a target. Acoustic liquid transfer machines, also referred to herein as acoustic liquid handlers, acoustic transfer apparatuses, and acoustic transfer devices, are a sub-category of liquid transfer machines used to perform accurate and precise direct, non-contact transfers of small (e.g., nanoliter) volumes of liquids between a source and a target without using pin tools, pipette tips, or washing. An acoustic liquid transfer machine accomplishes this direct, non-contact transfer of liquid by applying acoustic energy to a liquid source. When the acoustic energy is focused near the meniscus of the liquid, a mound of liquid is formed and a droplet is ejected, through the atmosphere, to a nearby target where it is captured and retained at the target by surface tension on the fluid. The diameter of the droplet scales inversely with the frequency of the acoustic energy. Thus, higher frequencies produce smaller droplets. For larger volumes, multiple droplets can be rapidly ejected from the source. This process of acoustic liquid transfer may be referred to as Acoustic Droplet Ejection (ADE) technology. Among other applications, acoustic liquid handlers are often used for high-throughput, automated workflows in the fields of pharmaceutical research, biotechnology, and diagnostics. Examples of suitable acoustic liquid transfer machines include, but are not limited to: the Echo® 650 Liquid Handler (Beckman Coulter), the Echo® 525 Liquid Handler (Beckman Coulter), the Echo® 655 Liquid Handler (Beckman Coulter), Echo® 550 Liquid Handler (Beckman Coulter), the Echo® 555Liquid Handler (Beckman Coulter), and the ATS Acoustic Transfer System (EDC Biosystems). Additional information related to liquid transfer, including acoustic liquid transfer, and be found, e.g., in U.S. Pat. Nos. 10,743,109 and 11,225,682, both of which are hereby incorporated by reference for such disclosures.

In some embodiments, the cells of the present disclosure are acoustically transferred in a high-density liquid medium (e.g., to improve the consistency of cell transfer and/or reduce cross-contamination). As noted above, the inventors of this disclosure surprisingly discovered that when cultured and incubated in standard media such as LB growth media, cells can become concentrated, e.g., sometimes in dense, circular pellets, at the bottom of the receptacle—leading to poor acoustic transfer. This is due to the nature of acoustic liquid transfer, the droplets ejected by the application of acoustic energy form at the meniscus/surface of the fluid. Thus, when the cells are not evenly dispersed throughout the fluid (e.g., are concentrated at the bottom), inconsistencies can arise in the number of cells transferred in each droplet and the transfer of cells can be poor and/or non-uniform. To improve the acoustic transfer of cells, provided are methods and compositions for using a high-density growth media prior to and during acoustic transfer to increase the buoyant force on the cells and reduce the rate of pelleting.

As noted above, a high-density growth media is growth media that includes components that increase the density of the growth media relative to standard growth medias, such as lysogeny broth (LB) (i.e., LB Medium, Luria Broth, Luria-Bertani medium). In some cases, a high-density growth media increases the buoyant force on the cells grown in the growth media and thereby reduces the rate of concentration of the cells at the bottom of the receptacle (e.g., pelleting). In some cases, a high-density growth media can include glycerol. In some cases, the high-density growth media includes a range of from 1-10% glycerol (e.g., 1-8%, 1-6%, 1-4%, 1-4%, 2-10%, 2-8%, 2-6%, 2-4%, 2-3%, 3-10%, 3-8%, 3-6%, 4-10%, 4-8%, 4-6%). In some cases, the high-density growth media includes a range of from 1-3% glycerol (e.g., 1%, 1.5%, 2%, 2.5%, or 3%). In some cases, the high-density growth media includes a range of from 3-7% glycerol (e.g., 3-5%, 4-6%, 4-5%). In some cases, the high-density growth media includes 2-3% glycerol. In some cases, the high-density growth media includes 1-3% glycerol. In some cases, the high-density growth media includes about 2.5% glycerol. In some cases, the high-density growth media includes about 5% glycerol. In some cases, a high-density growth media is can be, for example, [LB+2.5% glycerol (LGR), LB+5% glycerol (LGR), LB+2.5% glycerol (derived from Teknova LB+7.5% glycerol), LB+5% glycerol (derived from Teknova LB+7.5% glycerol)]. In some embodiments, the high-density growth media is LB with about 2.5% glycerol. Additional components in growth media formulations are known in the art and may include ingredients such as antibiotics, amino acids, glucose, vitamins, trace elements, and minerals. In some embodiments, the recipe for the high-density growth media is: 15 g tryptone, 7.5 g yeast extract, 7.5 g NaCl, 37.5 mL glycerol, 1462.5 mL water, and 100 g/mL carbenicillin.

In some embodiments, cells of the present disclosure are cultured in a high-density growth media. During acoustic transfer, acoustic energy is applied to the high-density growth media to form the droplet. As the cells are evenly dispersed throughout the growth media, the droplet contains the same (or similar) concentration of cells as the rest of the growth media, and cells are consistently and/or uniformly transferred to the target receptacle.

In some embodiments, the cells of the present disclosure are acoustically transferred when the cell culture is in early- to mid-log phase. Transfer of the cells when the culture is in early- to mid-log phase can reduce cross-contamination. Cells generally reproduce at a predictable, exponential rate that can be represented as a growth curve. During the log phase, also referred to as the exponential phase, cell growth is characterized by binary fission and doubling in population after each generation. Incubation conditions, such as temperature, rate of shaking, and length of incubation, are known in the art and may be adjusted to provide cells in early- to mid-log phase. For example, early- to mid-log phase may be achieved following about 10 hours to 12 hours of incubation at 37 degrees C. and 800 rpm. In another example, early- to mid-log phase may be achieved after about 16 hours to 18 hours of incubation at 30 degrees C. and 800 rpm. Other conditions for cell growth are known in the art and include, but are not limited to, oxygen, pH, light, osmotic pressure, atmospheric pressure, and moisture availability. Methods for determining when a cell culture has reached early- to mid-log phase are also known in the art.

### Copy Receptacle

In some embodiments, a clean copy of the acoustic transfer source receptacle (i.e., a clean copy of the automated colony selection destination receptacle) may be prepared prior to acoustic transfer of the cells from the source receptacle (e.g., prior to pooling). As the source and target receptacles are shifted and aligned for transfer of cells into specific locations, cross-contamination can occur as the source and target move past one another. To avoid cross-contamination caused by the traversal of the source and target past each other, cells from the source receptacle may first be acoustically transferred directly into the corresponding locations of an equivalent “copy” receptacle, thereby producing a copy of the source receptacle. As the cells are transferred only once, and only into the equivalent location on the copy receptacle, the source and target need only be aligned once to produce the copy, and the potential for cross-contamination is reduced. The copy may then be retained to preserve an uncontaminated set of cells arranged in the same configuration as the cells of the source receptacle, while the source receptacle can be used for additional acoustic transfer processes that have a higher likelihood of causing cross-contamination. In some embodiments, an equivalent receptacle is the same type of receptacle as the source receptacle. In other embodiments, the equivalent receptacle is a different type of receptacle than the source receptacle but contains the same subdivisions (i.e., the number of wells) as the source receptacle. In some cases, growth media may be added to the copy of the source receptacle either prior to or after the acoustic transfer of cells into the copy receptacle.

### Pooling

In some embodiments, the cells are acoustically transferred (from the automated colony selection ‘destination receptacle’, i.e., the acoustic transfer ‘source receptacle’) into pools of cells, such that cells originally located in separate locations (i.e., separate wells) or that have been otherwise separated (i.e., by colony) are combined into a common location, i.e., a pool. In such cases, the target receptacle can be referred to as a pooling receptacle. In some embodiments, cells in one location of the source receptacle may be transferred into one or more locations or one or more pools in the target receptacle. In some cases, the location of the pool in the target receptacle into which the cells are transferred is determined by a bitcode pooling scheme. Details of methods for determining the pools into which the cells are transferred are described elsewhere in the present disclosure.

Adjustments to acoustic liquid handlers can be made to further refine the acoustic transfer process, and in some cases reduce cross-contamination (e.g., via transfer of condensation droplets) into undesired locations of the target receptacle. These adjustments can include, but are not limited to, modifying the distance between the source and target receptacles (e.g., reducing or increasing), modifying the positions of the source and target receptacles relative to each other (e.g., aligning or offsetting), and modifying the timing between transfers (e.g., introducing a delay).

In some embodiments, the volume of liquid that is transferred by an acoustic liquid handler is in a range of from 1 nl to 100 μl (e.g., 1 nl to 50 μl, 1 nl to 10 μl, 1 nl to 1 μl, 1 nl to 750 nl, 1 nl to 500 nl, 2.5 nl to 100 μl, 2.5 nl to 50 μl, 2.5 nl to 10 μl, 2.5 nl to 1 μl, 2.5 nl to 750 nl, 2.5 nl to 500 nl, 10 nl to 100 μl, 10 nl to 50 μl, 10 nl to 10 μl, 10 nl to 1 μl, 10 nl to 750 nl, 10 nl to 500 nl, 100 nl to 100 μl, 100 nl to 50 μl, 100 nl to 10 μl, 100 nl to 1 μl, 100 nl to 750 nl, 100 nl to 500 nl, 250 nl to 100 μl, 250 nl to 50 μl, 250 nl to 10 μl, 250 nl to 1 μl, 250 nl to 750 nl, 250 nl to 500 nl, 500 nl to 100 μl, 500 nl to 50 μl, 500 nl to 10 μl, 500 nl to 1 μl, 500 nl to 750 nl). Said volume may be, e.g., about 1.0 nl, 2.5 nl, 5 nl, 7.5 nl, 10 nl, 15 nl, 20 nl, 25 nl, 30 nl, 35 nl, 40 nl, 45 nl, 50 nl, 58 nl, 60 nl, 70 nl, 80 nl, 90 nl, 100 nl, 110 nl, 115 nl, 122.5 nl, 130 nl, 140 nl, 150 nl, 160 nl, 170 nl, 180 nl, 190 nl, 200 nl, 205 nl, 210 nl, 220 nl, 230 nl, 240 nl, 250 nl, 260 nl, 270 nl, 280 nl, 285 nl, 300 nl, 350 nl, 400 nl, 450 nl, 500 nl, 520 nl, 540 nl, 572.5 nl, 600 nl, 700 nl, 800 nl, 900 nl, 1 μl, 1.5 μl, 2 μl, 2.5 μl, 3 μl, 4 μl, 5 μl, 6 μl, 7 μl, 8 μl, 9 μl or 10 μl. Said volume may be about 2.5 nl, 15 nl, 25 nl, 35 nl, 60 nl, 70 nl, 100 nl, 122.5 nl, 140 nl, 205 nl, 280 nl, 285 nl, 500 nl, 572.5 nl, 2 μl, or 10 μl. In some cases, additional liquid (e.g., growth media) can be added to the target receptacle following acoustic transfer of the cells.

The source receptacle may be any type of receptacle suitable for use with an acoustic liquid handler. In some cases, the source receptacle is a multi-well plate. In some embodiments, the source receptacle may have 6, 12, 24, 48, 96, 384, 1536, 3456 or any number of wells. In some embodiments, the source receptacle is further compatible for use with an automated colony picker. In some embodiments, the source receptacle is the automated colony selection destination receptacle. In some cases, where the automated colony selection destination receptacle is not compatible for use with an acoustic liquid handler, the contents of the automated colony selection destination receptacle are transferred to a receptacle suitable for use with an acoustic liquid handler prior to acoustic transfer. Examples of plates suitable for use as a source receptacle for acoustic transfer include, but are not limited, to Echo PP plates and Echo PP Plus plates.

The target receptacle may be any type of receptacle suitable for receiving cells transferred by an acoustic liquid handler. In some cases, the target receptacle is a plate comprising multiple wells. In some embodiments, the target receptacle may have 6, 12, 24, 48, 96, 384, 1536, 3456, or any number of wells. In some cases, where a copy of the source receptacle is being prepared, the target plate may be the same or have the same configuration as the source receptacle. Examples of plates suitable for use as a target receptacle for acoustic transfer include, but are not limited to, 384-well PCR places.

### Sequencing

Provided herein are methods and compositions that include nucleic acid sequencing (e.g., sequencing nucleic acids from pooled cells, e.g., cells pooled from predetermined wells of a source receptacle). In some embodiments, the nucleic acid sequencing is next generation sequencing (NGS). The term “Next Generation Sequencing (NGS)” herein refers to sequencing methods that allow for massively parallel sequencing of nucleic acid molecules. NGS technologies allow multiple samples to be sequenced individually (i.e., singleplex sequencing) or as pooled samples comprising indexed nucleic acids (e.g., multiplex sequencing) on a single sequencing run. Non-limiting examples of NGS include sequencing-by-synthesis using reversible dye terminators, and sequencing-by-ligation. In various embodiments analysis of the massive amount of sequence data obtained using NGS can be performed using one or more processors. More information related to NGS can be found, e.g., in U.S. Pat. No. 11,629,378, which is incorporated by reference herein for such disclosure.

### Sequencing Library Preparation

In some embodiments, the subject methods and compositions include preparing nucleic acid libraries for sequencing. Sequencing library preparation involves the production of a random collection of adapter-modified nucleic acid fragments (e.g., polynucleotides) that are ready to be sequenced.

In some cases, genetic material is first extracted from the cells. Extraction is performed to separate nucleic acids from other, unwanted cellular and sample matter to make the genetic material suitable for library construction. For example, this can be done with methods including, but not limited to, mechanical disruption (e.g., bead beating, sonicating, freezing and thawing cycles) and chemical disruption (e.g., by detergents, acids, bases, and enzymes). Isolation of the genetic material can be done through methods including, but not limited to, binding and elution from silica matrices, washing and precipitation by organic or inorganic chemicals, electroelution or electrophoresis, or other methods capable of isolating genetic material. More information can be found, e.g., in U.S. Pat. No. 11,492,672, which is incorporated by reference herein in its entirety.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originate in single-stranded form (e.g., ssDNA, RNA, etc.) and are converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation and may be known or unknown. In one embodiment, the polynucleotide molecules are DNA molecules.

For NGS libraries that are produced, the nucleic acid members can include a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM^{T}M and Ion Proton™ sequencing systems from Ion Torrent™; the PACBIO RS II Sequel system from Pacific Biosciences, the SOLiD sequencing systems from Life Technologies™, the 454 GS FLX+ and GS Junior sequencing systems from Roche, the MinION™ system from Oxford Nanopore, or any other sequencing platform of interest.

In some instances, the methods may include attaching sequencing platform adapter constructs to ends of a nucleic acid. For example, in some instances, oligonucleotides and/or primers utilized in the subject methods may not include sequencing platform adapter constructs and thus desired sequencing platform adapter constructs may be attached following the production of a nucleic acid of interest. Adapter constructs attached to the ends of a nucleic acid of interest or a derivative thereof may include any sequence elements useful in a downstream sequencing application. For example, the adapter constructs attached to the ends of nucleic acid of interest or a derivative thereof may include a nucleic acid domain or complement thereof selected from the group consisting of: a domain that specifically binds to a surface-attached sequencing platform oligonucleotide, a sequencing primer binding domain, a barcode domain, a barcode sequencing primer binding domain, a molecular identification domain, and combinations thereof.

Any suitable approach may be employed for providing additional nucleic acid sequencing domains to a nucleic acid of interest or derivative thereof having less than all of the useful or necessary sequencing domains for a sequencing platform of interest. For example, a nucleic acid of interest or derivative thereof could be amplified using PCR primers having adapter sequences at their 5′ ends (e.g., 5′ of the region of the primers complementary to the nucleic acid of interest or derivative thereof), such that the amplicons include the adapter sequences in the original nucleic acid as well as the adapter sequences in the primers, in any desired configuration. Other approaches, including those based on seamless cloning strategies, restriction digestion/ligation, tagmentation, or the like may be employed.

In certain embodiments, preparation of the next generation sequencing library may include amplification of all or a portion of the nucleic acids of the cells. In some cases, target specific amplification may be used to amplify specific nucleic acid sequences of interest. Such target specific amplification may make use of a target specific primer. By “target specific primer” is meant a primer that specifically hybridizes to a region of a nucleic acid sequence specific to the target of interest or the complement thereof. Amplification performed during library preparation, including e.g., target specific amplification, may be performed in a single round or multiple rounds of amplification may be employed. For example, in some instances, after a first round of amplification one or more amplification primers not utilized in the first round may be added to the reaction mixture to facilitate a second round of amplification using the product of the first round of amplification as a nucleic acid template.

In some instances, preparation of a library, e.g., a library for NGS, may include a step of purifying the nucleic acids and removing undesired nucleic acids or other contaminants within the sample and/or library. Any convenient method of purification may be employed including but not limited to e.g., nucleic acid precipitation (i.e., alcohol precipitation), gel purification, etc.

### Next-Generation Sequencing

Following prescribed library amplification steps, the prepared libraries may be considered ready for sequencing. In certain embodiments, the methods provided may further include subjecting a prepared sequencing library to an NGS protocol. The protocol may be carried out on any suitable NGS sequencing platform. NGS sequencing platforms of interest include, but are not limited to, a sequencing platform provided by Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD sequencing system); Roche (e.g., the 454 GS FLX+ and/or GS Junior sequencing systems); or any other sequencing platform of interest. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

### Sample Pooling and Deconvolution

In some embodiments, the cells, or the nucleic acids of the cells, of the present disclosure are pooled prior to sequencing. Following sequencing, the obtained nucleic acid sequences of the cell pools are deconvoluted such that a nucleic acid genotype can be assigned to individual colonies of cells.

### Sample Pooling

In some instances, samples of the present disclosure may be pooled prior to further processing and, as such, the performed methods may include a pooling step. For example, in some instances, cells originating from different colonies may be pooled together prior to preparation of one or more libraries. Any convenient method of pooling may be employed including, e.g., where entire sample volumes are pooled together or portions of sample volumes are pooled together. In some embodiments, cells from different colonies are pooled together using an acoustic liquid handler. Droplets containing cells of a single colony may be acoustically transferred from a source receptacle into pools configured to contain cells from a plurality of colonies in a target receptacle. In some embodiments, following colony selection, a portion of the cells separated by colony are removed prior to pooling to generate a clean copy that preserves the separation of cells by colony and reduces the potential for cross-contamination. In some embodiments, cells from a single colony may be mixed into one or more pools. In some embodiments, two or more pools may be combined together to form combined pools.

### Nucleic Acid Barcodes

In some instances, cells that are subsequently pooled may contain or be modified to contain an identifying nucleic acid sequence that allows for retrospective identification of the individual colony of the cell following pooling. Useful identifying nucleic acid sequences include, e.g., barcode nucleic acid sequences and indexing sequences.

In some instances, one or more barcode sequences may provide for retrospective identification of the source of a cell, e.g., following a sequencing reaction where the barcode is sequenced. For example, in some instances, a non-templated sequence that includes a barcode specific for the source (e.g., sample, well, colony, etc.) of the cell is incorporated during genetic modification of the cell. Such source identifying barcodes may be referred to herein as a “source barcode sequence” and such sequences may vary and may be assigned a term based on the source that is identified by the barcode. Source barcodes may include, e.g., a sample barcode sequence that retrospectively identifies the sample from which the cell was obtained, a well barcode sequence that retrospectively identifies the well (e.g., of a multi-well plate) from which the cell was obtained, a colony barcode sequence that retrospectively identifies the colony from which the cell was obtained, etc. Barcodes may find use in various procedures including, e.g., where cells are pooled following barcoding, e.g., prior to sequencing.

### Bitcode Sample Pooling Scheme

In some embodiments, the pools into which cells are deposited are determined using a bitcode sample pooling scheme. In a bitcode sample pooling scheme, each sample is assigned to and distributed amongst a different, specific subset of a plurality of pools. The pools into which a sample is distributed are referred to as the “sample bitcode” and can be represented as a series of 1s and 0s, in which a 1 represents a pool in which the sample is present, and a 0 represents a pool in which the sample is absent. After sequencing the nucleic acids in each pool, the obtained sequence data, the number of times each sequence is detected (referred to as the number of “reads”), and the pools in which those sequences are found can be deconvoluted to identify the sample from which the sequences originated. Sequences found to be present in a given subset of pools can be identified as originating from the sample distributed into those same pools. For example, in a 5-bitcode sample pooling scheme, five sample pools are prepared. Cells from a first colony are deposited in the Pools 1, 2, and 3. The sample bitcode for the first colony can be represented as [1, 1, 1, 0, 0]. Cells from a second colony are deposited in Pools 1, 4, and 5, and the sample bitcode is represented as [1, 0, 0, 1, 1]. Following sequencing, sequence A is found in Pools 1, 2, and 3 and can be attributed to cells from the first colony, while sequence B is found in Pools 1, 4, and 5 and can be attributed to cells from the second colony. Any number of pools may be used for a bitcode sample pooling scheme, such as from 5 to 48 pools, from 12 to 36 pools, or from 18 to 24 pools. In some embodiments, a 24-bitcode sample pooling scheme is used to determine the pools into which cells of the present disclosure are distributed.

### Deconvolution of Nucleic Acid Sequences

In some embodiments, deconvolution of nucleic acid sequences is performed using machine learning algorithms. Machine learning algorithms may be used to distinguish true genotype reads (i.e., reads of nucleic acid sequences across all pools that most likely reflect actual sequences detected or present in the pools) from spurious genotype reads (i.e., reads of nucleic acid sequences across all pools that most likely reflect PCR error, sequencing errors, cross-contamination, or other sequencing noise). In some instances, true genotype reads are characterized by both high counts in some pools, indicating that the sequence was introduced into and is present in a some particular pools, and by low counts (or zero counts) in other pools, indicating that, although a sequence may be present at very low levels due to error, the sequence was not intentionally introduced into those particular pools. In contrast, spurious genotype reads are generally characterized by excess zero and low read counts in all pools. Thus, when using a bitcode pooling scheme, a given sequence should have low read counts (or even zero counts) in some pools, but high read counts in other pools. The total number of reads for a given sequence across all pools can therefore be used to distinguish a true genotype (true sequence) from a spurious genotype (spurious sequence). As such, in some cases, machine learning is used to analyze the total number of reads for sequences from all pools—a threshold number of reads can be determined and used to distinguish true genotypes (true sequences) from spurious genotypes (spurious sequences).

Additionally, machine learning algorithms may also be used to binarize the data for each sequence from each pool. For each identified sequence, the machine learning algorithm may be used to determine whether the identified sequence is present or absent in each pool of the plurality of pools. If the identified sequence is present in a pool, the machine learning algorithm assigns a “1” for that pool. If the identified sequence is not present in a pool, the machine learning algorithm assigns a “0” for that pool. By assigning a “1” or a “0” to every pool for the identified sequence, the machine learning algorithm generates an experimental bitcode for the identified nucleic acid sequence. This experimental bitcode may then be matched against the sample bitcodes (i.e., the known subsets of pools into which a sample was distributed as determined by the bitcode sample pool scheme), thereby identifying the corresponding sample from which the nucleic acid sequence originated. In some cases, the originating sample is associated with a specific cell colony. In some cases, the originating sample is associated with a specific location on a receptacle (e.g., a specific well on a plate).

The machine learning algorithms may be unsupervised learning algorithms. Examples of unsupervised learning algorithms may include two-cluster Gaussian mixture models, artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering. In some embodiments, the machine learning algorithm is a two-cluster Gaussian mixture model (GMM).

The machine learning algorithms may be supervised learning algorithms. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, gradient boosted trees, Decision trees (e.g., C4.5, Random forests), Bayesian networks, and Hidden Markov models.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

The machine learning algorithm may be trained on a training dataset or datasets. The datasets may contain genotype reads from a sample or samples known to be true or spurious genotype reads. The datasets may contain reads representing nucleic acid sequences from a sample or samples in which the nucleic acid sequence is known to be present or absent.

### Curated Arrayed Libraries

Provided herein are methods and compositions for selecting cells to produce a curated arrayed library comprising cells that have selected nucleic acid sequences. In some embodiments, the nucleic acid genotype and the corresponding location of the cells comprising said genotype are employed to select and transfer cells from the automated colony selection destination receptacle (i.e., the acoustic transfer source receptacle) or a copy receptacle (i.e., a copy of the automated colony selection destination receptacle) to a new receptacle to produce a curated arrayed library of cells.

### Selection of Samples

In some embodiments, following deconvolution and identification of the each of the samples from which the nucleic acid sequences originated, or the identification of the locations of a receptacle containing each of the samples, certain samples may be selected, and the cells from each sample may be removed and placed into a separate location of a receptacle to produce a curated arrayed library. Various criteria may be used to select the samples. In some cases, samples that are predicted to include more than one of a unique nucleic acid sequence of interest (i.e., multiple unique nucleic acid sequences of interest were predicted to be in a single location of the destination receptacle/in a single colony of cells where each should have only one unique nucleic acid sequence of interest) are undesirable and should not be selected. In other cases, when the same unique nucleic acid sequence of interest is predicted to be present in multiple samples (i.e., multiple locations of the destination receptacle/multiple colonies each contain the same unique nucleic acid sequence of interest), only one of these samples should be selected, and the rest are undesirable. As such, in some embodiments, samples that should be selected are those that contain only one nucleic acid sequence of interest and do not share the nucleic acid sequence of interest with another selected sample.

### Producing a Curated Arrayed Library

In some embodiments, the cells from the selected colonies are removed and placed into a new receptacle to produce a curated arrayed library. In some embodiments, the cells are transferred to the curated arrayed library using acoustic transfer. In some instances, the cells are removed from samples contained in the acoustic transfer source receptacle (i.e., the automated colony selection destination receptacle). In some embodiments, the cells are removed from the copy receptacle (i.e., the copy of the acoustic transfer source receptacle)(i.e., the copy of the automated colony selection destination receptacle). In some instances, additional growth media is added to the curated arrayed library following acoustic transfer of the cells.

The curated arrayed library may be used for any number of downstream applications or workflows. For example, the cells in the curated arrayed library may be used to produce larger culture volumes of the cells. The larger cultures may subsequently be used to, for example, prepare glycerol stocks, obtain plasmids, generate lentiviruses, perform functional screens, and the like.

### Computers

Also provided are one or more computational systems (e.g., a computer) that may be used in the methods and compositions of the present disclosure. In some embodiments, a computational system of the present disclosure may be used to control the automated colony picker, including, but not limited to, receiving input to select colonies (e.g., from the user, or by using automated image analysis algorithms to identify and select colonies), controlling the automated apparatus, and making adjustments to and controlling the movement and position of the pins, source receptacle, and/or the destination receptacle. In some embodiments, a computational system of the present disclosure may be used to control the acoustic liquid handler, including, but not limited to, receiving input regarding the locations from which liquids should be transferred and the locations in which the liquids should be deposited, making adjustments to and controlling the movement and position of the source and/or destination receptacle, calculating the acoustic energy required to eject a droplet of a desired size, and controlling the characteristics (e.g., wavelength, amplitude, frequency, time period, and velocity) and application of the acoustic energy to the liquid source. In some embodiments, a computational system of the present disclosure may be used to control additional robotics devices configured to perform actions including, but not limited to, adding growth media to source and destination receptacles and delivering and moving source and destination receptacles to a device (e.g., automated colony picker, acoustic liquid handler). In some embodiments, a computational system of the present disclosure may be configured to analyze and deconvolute nucleic acid sequencing data. Each computational system of the present disclosure may perform one or more functions as described above.

A computational unit may include any suitable components to perform the functions as described above. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

Raw data, such as images of colony plates, the number of reads for each nucleic acid sequence, and the like, can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present disclosure. The data storage means may comprise any manufacture comprising a recording of the present information as described above, or a memory access means that can access such a manufacture.

Performance of the described functions may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of the present disclosure. In some embodiments, the function is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

The computer also comprises a program of instructions. The program of instructions can comprise a machine learning algorithm used for image analysis and/or the analysis and deconvolution of the obtained nucleic acid sequences as described above. Any machine learning algorithms deemed useful may be used. Useful machine learning algorithms include, without limitation, two-cluster Gaussian mixture models, logistic regression, random forest, gradient boosted tree, support vector machine, linear/quadratic discriminant analysis, k nearest neighbors, nave bayes, neural network, etc.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The data and analysis thereof can be provided in a variety of media to facilitate their use. “Media” refers to a manufacture that contains the signature pattern information of the present disclosure. The data of the present disclosure can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present data and information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

Further provided herein is a method of storing and/or transmitting, via computer, sequence, and other, data collected by the methods disclosed herein. Any computer or computer accessory including, but not limited to software and storage devices, can be utilized to practice the present disclosure. Sequence or other data can be input into a computer by a user either directly or indirectly. Additionally, any of the devices which can be used to sequence DNA or analyze DNA can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### Utility

The methods and compositions provided herein facilitate the ability to rapidly generate arrays of genotypically identified cells from an initial starting pool. As one example, these arrays can be composed of a diverse set of E. coli expressing lentivirus expression plasmids encoding CRISPR guide RNAs targeting a diverse set of genomic loci.

The methods and compositions described herein can be used for high-throughput generation of genotypically identified clones, in some cases with a throughput, e.g., of approximately 8,000 clones per 3 week cycle. With this throughput, a custom genome-wide library targeting all 20,000 protein coding genes of the human genome could be generated in approximately two months. In contrast, previously described methods, such as colony picking followed by Sanger sequencing, or colony picking follow by row and column deconvolution, can typically take upwards of 1 or more years to complete. Production of these types of CRISPR libraries facilitates on-demand knockdown, knock-up, and knockout of any gene in the human genome. Additionally, the methods and compositions described herein facilitate quick and facile generation of novel libraries of interest that may arise. These include those using different CRISPR modalities (e.g. base editing, CRISPR transposons) as well as expanded sets of genes of interest (e.g. enhancers, non-coding RNAs).

Additionally, aspects of the methods and compositions described herein may also find application in diagnostic screening. An example of this is high-throughput screening of patient samples for specific microorganismal DNA/RNA sequences. In this application, the patient samples can be loaded into multiwell plates, combinatorially pooled, sequenced, and computationally deconvolved to identify samples possessing specific nucleotide sequences of interest.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method of creating an arrayed library of cells, comprising:
   a) performing automated colony selection, comprising:
      i) selecting colonies of cells from a colony source and depositing each of the colonies into a unique location of a first receptacle using an automated apparatus for automatic handling of one or more pick tools, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle; and
   b) pooling the cells from the selected colonies, comprising:
      i) acoustically transferring, using an acoustic liquid handler, a portion of the cells from each said unique location of the first receptacle, wherein the first receptacle contains a high-density media, to one or more locations of a second receptacle to produce cell pools;
   c) sequencing nucleic acids of the cell pools to obtain nucleotide sequences; and
   d) deconvoluting the obtained nucleotide sequences to assign a nucleic acid genotype to each unique location of the first receptacle.
2. The method of 1, wherein the cells are genetically modified.
3. The method of 2, wherein the genetically modified cells comprise members of a molecular library.
4. The method of 3, wherein the molecular library comprises a plurality of nucleic acids comprising one or more nucleic acid barcode sequences.
5. The method of 3 or 4, wherein the molecular library is a guide RNA library, a transgene library, an shRNA library, a long noncoding RNA library, an open reading frame expression library, a library of mutated nucleic acid sequences, a library of viral nucleic acid sequences, or any combination thereof.
6. The method of 3 or 4, wherein the molecular library comprises vectors and each of the vectors comprises one or more guide RNAs.
7. The method of 2-6, wherein the cells comprise a genetic modification introduced by transformation, transduction, or transfection.
8. The method of 7, wherein the cells are virally transduced.
9. The method of any one of 1-8, wherein the cells are microorganisms.
10. The method of 9, wherein the microorganisms are bacteria, parasites, or fungi.
11. The method of 9, wherein the microorganisms are bacteria.
12. The method of any one of 1-11, further comprising producing the colonies of cells.
13. The method of any one of 1-12, wherein the automated apparatus for automatic handling of one or more pick tools is a Hudson Robotic RapidPick MP.
14. The method of any one of 1-13, wherein the automated apparatus for automatic handling of one or more pick tools is modified to reduce the speed at which the active pick tool is extended and retracted during colony selection.
15. The method of any one of 1-14, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.
16. The method of any one of 1-15, wherein the first receptacle is a multi-well plate suitable for use with an acoustic liquid handler.
17. The method of any one of 1-16, wherein the high-density media comprises LB broth and 1-10% glycerol.
18. The method of any one of 1-16, wherein the high-density media comprises about 2.5% glycerol.
19. The method of any one of 1-18, wherein the high-density media is added to the first receptacle prior to performing automated colony selection.
20. The method of any one of 1-19, wherein the one or more locations of the second receptacle into which the cells are transferred from the first receptacle are determined using a bitcode sample pooling scheme.
21. The method of 20, wherein the bitcode sample pooling scheme is a 24-bitcode sample pooling scheme.
22. The method of any one of 1-21, wherein the acoustic liquid handler is an Echo 525 Liquid Handler.
23. The method of any one of 1-22, wherein the acoustic transfer occurs when the cells are in early- to mid-log phase.
24. The method of any one of 1-23, wherein deconvoluting the obtained nucleic acid sequences comprises using an unsupervised machine learning algorithm.
25. The method of 24, wherein the machine learning algorithm is a Gaussian Mixture Model algorithm.
26. The method of any one of 1-25, further comprising employing the nucleic acid genotype and associated unique location of the first receptacle to select and transfer cells from the first receptacle to a third receptable, thus producing a curated arrayed library of cells.
27. The method of 26, further comprising preparing a copy of the first receptacle following automated colony selection and prior to pooling the cells.
28. The method of any one of 1-25, further comprising preparing a copy of the first receptacle following automated colony selection and prior to pooling the cells, thus producing a copy receptacle.
29. The method of 28, further comprising employing the nucleic acid genotype assigned to each unique location of the first receptacle to select and transfer cells from the copy receptacle to a third receptable, thus producing a curated arrayed library of cells.
30. A system for selecting and transferring cells, comprising:
   a) an automated apparatus for automatic handling of one or more pick tools and configured for performing automated selection of colonies of cells into one or more locations of a first receptacle, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle;
   b) the first receptacle, comprising a high-density media;
   c) an acoustic liquid handler configured to acoustically transfer a portion of the cells from the first receptacle to one or more locations of a second receptacle.
31. The system of 30, further comprising the cells.
32. The system of 31, wherein the cells are genetically modified.
33. The system of 32, wherein the genetically modified cells comprise members of a molecular library.
34. The system of any one of 30-33, wherein the automated apparatus for automatic handling of one or more pick tools is a Hudson Robotic RapidPick MP.
35. The system of any one of 30-34, wherein the automated apparatus for automatic handling of one or more pick tools comprises a modification to reduce the speed at which the active pick tool is extended and retracted during colony selection.
36. The system of any one of 30-35, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.
37. The system of any one of 30-36, wherein the first receptacle is a multi-well plate suitable for use with an acoustic liquid handler.
38. The system of any one of 30-37, wherein the high-density media comprises LB broth and 1-10% glycerol.
39. The system of any one of 30-37, wherein the high-density media comprises about 2.5% glycerol.
40. The system of any one of 30-39, further comprising the second receptacle.
41. The system of any one of 30-40, wherein the acoustic liquid handler is an Echo 525 Liquid Handler.
42. The system of any one of 30-41, further comprising a computer configured for deconvolution of nucleic acid sequences.
43. The system of 42, wherein the computer is configured for deconvolution using an unsupervised machine learning algorithm.
44. The system of 43, wherein the machine learning algorithm is a Gaussian Mixture Model algorithm.
45. A method of selecting and transferring cells, comprising:
   a) performing automated colony selection, comprising:
      i) selecting colonies of cells from a colony source and depositing the colonies in one or more locations of a first receptacle using an automated apparatus for automatic handling of one or more pick tools, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle; and
   b) acoustically transferring, using an acoustic liquid handler, a portion of cells from the first receptacle, wherein the first receptacle contains a high-density media, to one or more locations of a second receptacle.
46. The method of 45, wherein the automated apparatus for automatic handling of one or more pick tools is a Hudson Robotic RapidPick MP.
47. The method of 45 or 46, wherein the automated apparatus for automatic handling of one or more pick tools is modified to reduce the speed at which the active pick tool is extended and retracted during colony selection.
48. The method of any one of 45-47, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.
49. The method of any one of 45-48, wherein the high-density media is added to the first receptacle prior to performing automated colony selection.
50. The method of any one of 45-49, wherein the high-density media comprises about 2.5% glycerol.
51. The method of any one of 45-50, wherein the acoustic liquid handler is an Echo 525 Liquid Handler.
52. The method of any one of 45-51, wherein the acoustic transfer occurs when the cells are in early- to mid-log phase.
53. A method of acoustically transferring cells, comprising:
   a) obtaining a first receptacle comprising one or more cells in a high-density media; and
   b) acoustically transferring, using an acoustic liquid handler, at least one of said one or more cells in the high-density media from the first receptacle to one or more locations of a second receptacle.
54. The method of 53, wherein the high-density media comprises about 2.5% glycerol.
55. The method of 53 or 54, wherein the acoustic liquid handler is an Echo 525 Liquid Handler.
56. The method of any one of 53-55, wherein the acoustic transfer occurs when the cells are in early- to mid-log phase.
57. A system for acoustically transferring cells, comprising:
   a) one or more cells in a first receptacle comprising a high-density media;
   b) an acoustic liquid handler configured to acoustically transfer at least one of said one or more cells from the first receptacle to one or more locations of a second receptacle.
58. The system of 57, wherein the high-density media comprises about 2.5% glycerol.
59. The system of 57 or 58, wherein the acoustic liquid handler is an Echo 525 Liquid Handler.
60. The system of any one of 57-59, wherein the cells are in early- to mid-log phase.
61. A method for reducing contamination during automated colony selection, comprising selecting colonies of cells from a colony source and depositing the colonies in one or more locations of a destination receptacle using an automated apparatus for automatic handling of one or more pick tools, wherein the automated apparatus comprises a solid barrier comprising an opening for an active pick tool to access the colony source and the destination receptacle, and wherein the solid barrier is mounted on the automated apparatus and configured to prevent transfer of cells from an inactive pick tool into an undesired location of the destination receptacle.
62. The method of 61, wherein the automated apparatus for automatic handling of one or more pick tools is a Hudson Robotic RapidPick MP.
63. The method of 61 or 62, wherein the automated apparatus for automatic handling of one or more pick tools is modified to reduce the speed at which the active pick tool is extended and retracted during colony selection.
64. The method of any one of 61-63, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.
65. A solid barrier mounted on an automated apparatus for automatic handling of one or more pick tools, wherein
   a) the pick tools are configured to select colonies of cells from a colony source and deposit the colonies in one or more locations of a destination receptacle;
   b) the solid barrier comprises an opening for an active pick tool to access the colony source and the destination receptacle; and
   c) the solid barrier is configured to prevent transfer of cells from an inactive pick tool into an undesired location of a destination receptacle.
66. The solid barrier of 65, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.
67. The solid barrier of 65 or 66, wherein the automated apparatus for automatic handling of one or more pick tools is a Hudson Robotic RapidPick MP.
68. The solid barrier of any one of 65-67, wherein the automated apparatus for automatic handling of one or more pick tools comprises a modification to reduce the speed at which the active pick tool is extended and retracted during colony selection.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like

### Example 1: Generation of Arrayed Libraries

### Generate Library of Host Cells

In this particular example, E. coli expressing Cas9 guide RNA lentivirus transfer plasmids were used. In other words, the molecular library was guide RNA sequences encoded by lentivirus plasmids and the host cells were E. coli cells. The goal was to create a genotypically-defined arrayed library of E. coli cells.

Barcode Cloning into Dual Guide Expression Vector (Expression Vector Encoding Two Guide RNAs)

To diversify the vector backbone to aid in deconvolution, we ordered oligos containing 96 different 11 bp barcodes (min. Hamming dist.=3). These barcodes were cloned into the XhoI/BamHI site 3′ to the transcription termination site of PS2 (“PS” is protospacer, and is intended to mean the targeting sequence of the guide RNA). The cloning product was transformed into high efficiency DH5α, plasmid purified, and re-transformed into subcloning efficiency DH5α. A pilot run of combinatorial-pooling based arrayed library generation was used to recover 42 unique barcoded vectors. Plasmids produced from these vectors were quantified and normalized to produce a pool of dual guide vectors containing 42 unique barcodes.

Dual Guide Cloning into Barcoded Dual Guide Expression Vector

Oligos containing PS1 and PS2 (guide RNA targeting sequence 1 and guide RNA targeting sequence 2) were ordered in pooled format. Oligos were PCR amplified and cloned into the BlpI/BstXI sites of a normalized pool of 42 barcoded dual guide vectors. The cloning product was transformed into high-efficiency DH5α and plasmid purified. The CR3-hU6 insert was cloned into the BsmBI sites of the PS1/PS2-containing vectors and the cloning product transformed into high-efficiency DH5α and plasmid purified. A secondary BsmBI digestion was performed to digest background vector. Restriction digest product was transformed into high-efficiency DH5α and plasmid purified to produce a library of fully assembled barcoded dual guide vectors.

E. coli Transformation

E. coli were transformed into subcloning efficiency DH5α and plated onto appropriate media plates compatible with the selected colony picker. In this case, 86×128 mm LB agar plates containing 100 μg/mL carbenicillin (Teknova L2010) were used (the E. coli transfer plasmids expressed carbenicillin resistance). Plates were incubated to obtain the desired colony size (in this case 16 h at 37 C).

### Automated Colony Picking

In this particular embodiment, an automated colony picking protocol was used with a Hudson Robotics Colony Picker. The protocol used automated image analysis algorithms to identify colonies and positioned the media plate for colonies to be picked by the robotic pins. For high-throughput processing, the Hudson Robotics Colony Picker was integrated into a larger robotics system that delivers media plates containing microorganisms (source plate) to the picker, fills and delivers multi-well plates (destination plates) with appropriate media (in this case a high-density media: LB+2.5% glycerol), and moves source and destination plates out of tower stacks as media plates are depleted and destination plates are filled. To help differentiate between plates produced at different steps, plates produced in this step are termed “ALL-” plates.

After picking was completed, destination plates were sealed and incubated to achieve desired culture density for Echo-based acoustic transfer. In this particular embodiment, the plates were incubated for 16 h and 30 C, 800 rpm.

After the desired incubation time, “ALL-” plates were removed from the incubator and allowed to come to room temperature for 1 h (The Echo 525 is designed to acoustically transfer liquids at room temperature/25 C).

### Copy Plate Generation

Due to inter-well contamination produced during combinatorial pooling on the Echo 525, prior to combinatorial pooling, 1:1 full copies of “ALL-” plates were generated. These copies were termed “CPY-” plates. These “CPY-” plates served as clean, non-contaminated source material for the cherry picking step of the process (i.e., generating a curated library). “CPY-” plates were stored at 4 C awaiting cherry picking.

### Combinatorial Pooling (According to Predetermined Bitcode)

“ALL-” plates containing one microorganism colony per well were combinatorially pooled following pre-defined 24-bitcodes using 384-well PCR plates as the destination plates (1 column per bit of a 24-bitcode). For example, if bitcode “001010001101010111100010” is assigned to Well A1 of Plate 1, the Echo 525 would be used to acoustically transfer droplets from this well into columns 3, 5, 9, 10, etc. of the destination plate (“1” bits), but would not transfer droplets into columns 1, 2, 4, 6, 7, etc. (“0” bits). Each well of each “ALL-” plate was transferred using a unique 24-bit pooling bitcode. The set of pooling bitcodes employed encompassed 13,000 bitcodes with a minimum Hamming distance of 6 between bitcodes, allowing theoretical generation of genotypic predictions on up to 13,000 samples within a single run.

The small pools generated in the 384-well pooling plates were pooled into 24 final pools (1 for each bit of a 24-bitcode) using an automated method on the Hamilton STAR. This step was used because during transfer on the Echo 525, the destination plate was inverted and had a maximum retention volume per well of approximately 15 uL. As we combinatorially pooled clones, once Plate 1, Row A reached 15 uL, we moved to subsequent rows and plates until all clones have been combinatorially pooled. We then use the Hamilton STAR to “add up across rows” to generate final pools where Pool 1 contained all clones with a “1” bit in the 1^{st} location of their 24-bitcode, Pool 2 contained all clones with a “2” bit in the 2^{nd} location of their 24-bitcode, etc.

### Library Preparation and Sequencing

NGS libraries were generated from the 24 pools generated in previous step. This involved plasmid miniprep from the 24 E. coli pools, followed by PCR amplification where each pool received a unique index to facilitate multiplexed sequencing.

For sequencing, a sufficiently large number of sequencing reads were produced to distinguish signal (“1” bits) from noise (“0” bits). In our hands, sequencing on a NextSeq 550 using a Hi Output kit typically produced sufficient read depth.

### Computational Deconvolution and Microorganism Genotype Assignment (Prediction)

NGS data were analyzed through a computational script that identifies genotypic sequences observed in each of the 24 pools, automatically identifies a read count threshold distinguishing “1” bits from “0” bits through use of sequential two-cluster Gaussian mixture models, and then generates genotype predictions for wells of the “ALL-”/“CPY-” plates. These predictions were used to generate a cherry pick list (a list of wells to select from for generating a curated library).

### General Overview of the Steps of the Computational Deconvolution Pipeline

In brief, reads observed in 24 pools are tabulated across each unique genotype observed. An example may look like this: GCTACGCCCGGGGGAAAAGA_GCAGGAGCTAAGGGTCCCGT_ACCAGCCGATG: [1, 3, 37305, 0, 5, 26502, 20, 56173, 6, 3, 41560, 39019, 2, 42514, 12, 5, 29151, 61658, 38040, 31852, 29176, 16, 21, 19468] (SEQ ID NO: xx)

The high counts correspond to the microorganism containing this genotype being transferred into this pool (Pools 3, 6, 8, etc. in this case) while the low counts correspond to the microorganism containing this genotype not being transferred into this pool (Pools 1, 2, 4, 5, etc. in this case). Rather than non-transfers appearing as zero counts, they often appear as low counts, due to a combination of PCR/sequencing errors and potentially mild rates of cross-contamination between pools. The challenge in deconvolution lies in converting the raw counts data into a binarized “experimental NGS bitcode” that can then be matched to the original bitcodes used for pooling, leading to a genotype prediction in specific wells. Binarization is accomplished through sequential application of two-cluster Gaussian mixture models (GMM). In the first GMM, the log 2(sum) of all 24 reads counts per genotype (1 sum per genotype) is plotted on the x-axis. This is intended to separate “true” genotype reads from “spurious” genotype reads that arise from sequencing noise, as “spurious” reads arbitrarily introduce excess zero and low read counts, rendering it more difficult to identify a robust threshold separating high and low read counts of “true” genotype reads.

A “true” genotype read is one believed to arise from a real sample, and is characterized by a mixture of high and low read counts. An example of a “true” genotype read is: GCTACGCCCGGGGGAAAAGA_GCAGGAGCTAAGGGTCCCGT_ACCAGCCGATG: [1, 3, 37305, 0, 5, 26502, 20, 56173, 6, 3, 41560, 39019, 2, 42514, 12, 5, 29151, 61658, 38040, 31852, 29176, 16, 21, 19468] (SEQ ID NO: xx)

A “spurious” genotype read is one believed to arise from sequencing noise, and is characterized by exclusively low counts. An example of a “spurious” genotype read is: GGGAGGTACCGGCTGTTGTG_GGGCAGAGCCGCACAACAGC_AGATTCCGCCC: [0, 0, 0, 1, 0, 3, 0, 0, 0, 0, 0, 0, 0, 0, 0, 0, 0, 0, 0, 0, 0, 0, 0, 3] (SEQ ID NO: xx)

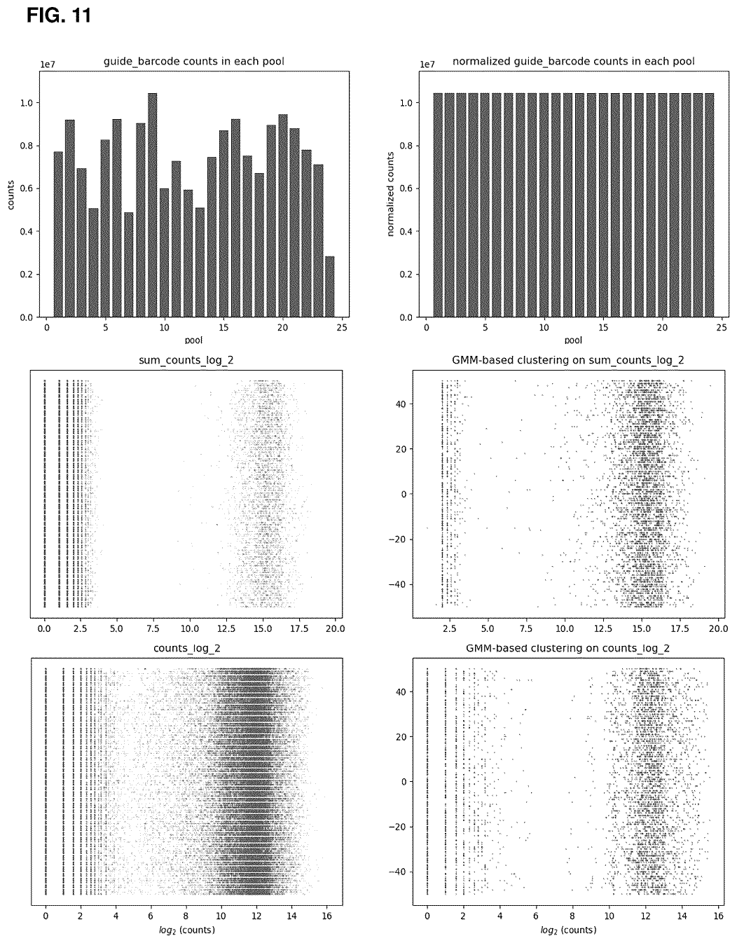

An example of raw data for this GMM is found in Row 2, Left Panel of FIG. 11, while the clustering results are found in Row 2, Right Panel of FIG. 11, with the left cluster representing “spurious” reads, and the right cluster representing “true” reads. Only data from the “true” reads cluster is used in the second GMM for distinguish the read counts threshold for binarization of each individual count.

In the second GMM, all log 2(counts) of all individual read counts (24 counts per genotype) are plotted. An example of raw data from this GMM is found in Row 3, Left Panel of FIG. 11, while the clustering results are found in Row 3, Right Panel of FIG. 11, with the left cluster composed of low counts that will be converted to “0” bits, and the right cluster composed of high counts that will be converted to “1” bits. Sufficient signal-to-noise for distinguishing clusters is visually indicated by a bifurcation in the plot, with low scatter density between the two clusters.

Counts data of each “true” genotype read is then binarized according to this clustering algorithm. An example of the binarized version of the “true” genotype read above is:

GCTACGCCCGGGGGAAAAGA_GCAGGAGCTAAGGGTCCCGT_
ACCAGCCGATG:
‘001001010011010011111001’.

This “experimental NGS bitcode” is then matched to the pooling bitcodes used during combinatorial pooling on the Echo 525, with matches leading to genotype predictions. In this case, the bitcode ‘001001010011010011111001’ was used to combinatorially pool Plate 1, Well A3, leading to the prediction that the E. coli in Plate 1, Well A3 has the genotype:

GCTACGCCCGGGGGAAAAGA_GCAGGAGCTAAGGGTCCCGT_
ACCAGCCGATG

### Producing a Curated Library

Once genotypic predictions were completed and desired microorganism clones identified for cherry-picking, “CPY-” plates were removed from 4 C and incubated for 12 h at 30 C, 800 rpm to produce the desired culture density for cherry-picking on the Echo 525.

Desired clones were cherry-picked on the Echo 525 using a picklist generated in the previous step. Cherry-picking involved transferring select clones from the “CPY-” plates into “cherry-picked” (“CHR-”) plates to generate a dense collection of desired clones (a curated arrayed library). Clones in the “CHR-” plates could then be used in downstream workflows. In our hands, these cultures were inoculated into larger culture volumes, from which glycerol stocks and plasmid minipreps were produced. These plasmids can then be used to generate lentivirus for delivering Cas9 CRISPR guide RNA constructs into target cell lines for arrayed functional screens.

Experimentation that LED to the Above

### Explanation

In initial experiments, during colony picking, E. coli clones were picked into standard LB media. We observed that post-incubation, cultures would develop a pellet, with E. coli concentrated in dense, circular pellets at the bottom of each well. During Echo 525-based acoustic transfer of these cultures, we observed inconsistent transfer of E. coli into the destination plate. We determined this was likely due to the nature of acoustic transfers on the Echo 525, in which droplets generated at the meniscus are transferred upwards into a destination plate. The pelleting observed in LB media concentrated most of the E. coli at the bottom of the well, leaving a sparse culture of E. coli in solution. This led to inconsistent and variable transfer of E. coli. The combinatorial pooling and deconvolution approach employed herein is facilitated by approximately uniform E. coli transfer of each well in the “ALL-” plate to establish an accurate NGS read count threshold differentiating counts corresponding to “1” bits and “0” bits. Due to the importance of achieving uniform E. coli transfer, we experimented with a number of different solutions. We experimented with culturing the “ALL-” plates for a shorter period of time prior to combinatorial pooling, as well as briefly vortexing the “ALL-” plates prior to combinatorial pooling. These approaches either did not work or were not high-throughput/reliable. We then experimented with higher density media formulations, based on the idea that a higher density media would increase the buoyant force on E. coli, potentially reducing the rate of pelleting.

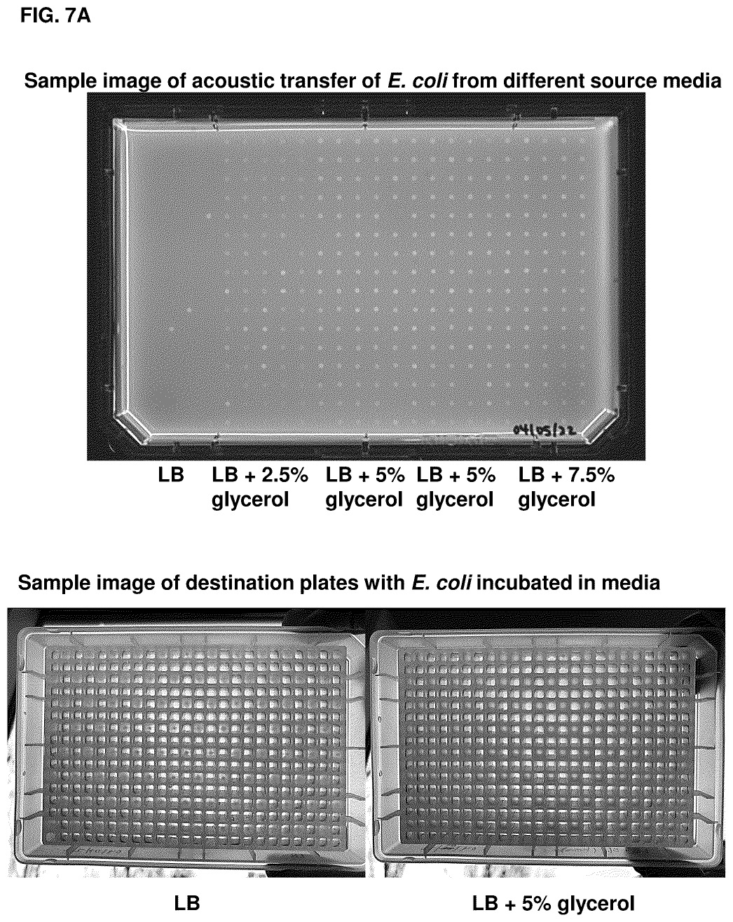

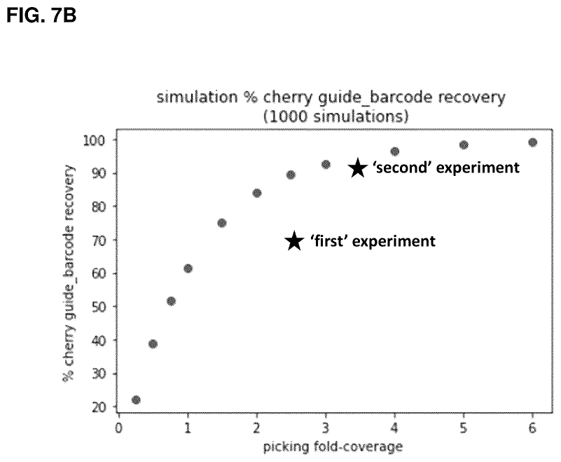

In total, 10 different media formulations were tested, and we selected LB+2.5% glycerol (10 g tryptone, 5 g yeast extract, 5 g NaCl, 25 mL glycerol, 975 mL H2O) as our final (best) media, based on its ability to support E. coli growth and contamination-free colony picking and acoustic transfer. All mentions of “pelleting” below and the use of a higher density media are intended to address non-uniform transfer of E. coli samples from source plates (e.g., to target plates) using the Echo 525. These transfers are important in combinatorial pooling as well as cherry-picking. Sample images of E. coli incubated in LB/LB+5% glycerol, and transfer of E. coli grown in these media can be found in FIG. 7A.

### Automated Colony Picking

Echo PP Plates (Beckman Coulter #PP-0200) were pre-filled with 40 μL LB (Teknova #L8050). Colonies plated onto LB agar carbenicillin plates were picked into individual wells using a Hudson Robotics RapidPick MP following a “wide checkerboard” pattern. This pattern was chosen to assess the rate of cross-contamination by identification of growth in wells that were not picked into. We identified high rates of cross-contamination in the right portions of the plate (Columns 18+) and hypothesized this was due to the architecture of the colony picker, where pins that had delivered E. coli but had not yet been sterilized temporarily hovered over this region of the plate. To address this issue, we designed a shield with a single hole for delivery of E. coli to the destination plate. The shield was designed such that pins that had delivered E. coli but had not yet been sterilized hovered over the shield rather than exposed regions of the plate. Several prototypes of the shield were designed and laser cut prior to the final design. Once the final design is in plate, we no longer observed cross-contamination when colony picking into Echo PP Plates pre-filled with 40 μL LB.

After E. coli were picked into the Echo PP Plates, plates were incubated for 24 h at 37 C, 800 rpm in an Infors HT Multitron shaking incubator. After incubation, pelleting of E. coli within individual wells was observed.

To resuspend the E. coli, individual Echo PP Plates were vortexed on a Vortex-Genie 2 Lab Mixer. Resuspension did not appear successful, and splashing was observed on the seal.

To address E. coli pelleting, higher density media formulations were designed with the goal of increasing the buoyant force acting on E. coli. In total, 10 different media formulations were designed and tested: 1) LB; 2) LB+5% glycerol (LGR recipe); 3) Teknova Super Broth; 4) Teknova Super Broth+5% glycerol; 5) 2× Teknova Super Broth; 6) 2× Teknova Super Broth+5% glycerol; 7) 2× Teknova Super Broth+5% PEG8000; 8) 3× Teknova Super Broth; 9) 3× Teknova Super Broth+5% glycerol; 10) 3× Teknova Super Broth+5% PEG8000.

For each media formulation, 4 mL of media was inoculated with 2 uL starter culture and incubated for 14.5 h at 37 C, 250 rpm. The OD600 of each culture was measured, and each culture pelleted and miniprepped. Observations regarding culture appearance, OD600, pellet size, and miniprep DNA quality were taken. Media formulations #4-10 did not support E. coli growth and/or plasmid recovery and were nor pursued further.

To test transfer properties of media formulations #1-3, an Echo PP Plate was pre-filled with these media formulations, with media formulation #1 in Columns 1-8, media formulation #2 in Columns 9-16, and media formulation #3 in Columns 17-24. E. coli was transformed and plated onto an LB agar carbenicillin plate incubated at 37 C for 16 h in a static incubator. Colonies were picked into each well of the pre-filled Echo PP Plate and incubated for 24 h at 37 C, 800 rpm in a shaking incubator. The Echo PP Plate was removed from the incubator and allowed to come to RT for 1 h. An Echo 525 was used to make a full copy of the Echo PP Plate onto an LB agar carbenicillin plate using 25 nL transfers per well. The LB agar carbenicillin plate was incubated for 14 h at 30 C in a static incubator.

Of the three media formulations, we observed that LB+5% glycerol produced the largest colony size (e.g. highest #E. coli transferred). We decided to pursue this media formulation for further development. Due to the change in the media formulation, we began to use Echo PP Plus Plates (Beckman Coulter #PPL-0200) rather than Echo PP Plates as the destination plates for colony picking.

E. coli was transformed and plated onto LB agar carbenicillin plates and incubated at 37 C for 16 h in a static incubator. Echo PP Plus Plates were pre-filled with 40 μL LB+5% glycerol (LGR) and E. coli colonies were picked into individual wells using a “wide checkerboard” pattern (A1, A3, . . . , A23, C2, C4 . . . , C24, E1, E3 . . . ). Using the LB+5% glycerol (LGR) media formulation, we observed extensive cross-contamination after culturing.

We hypothesized that quick entry and exit of the pin from the destination wells may have produced splashing or carryover of E. coli, resulting in cross-contamination. Hardware adjustments were made to the colony picker to reduce air pressure to the pistons driving pins for E. coli picking and placement. This effectively reduced the speed of entry and exit of the pin when delivering E. coli to pre-filled Echo PP Plates. After hardware adjustments, we observed a decrease in rates of cross-contamination. The cross-contaminations, however, were not completely resolved.

To further decrease the rate of cross-contamination, we designed and tested additional alternative media formulations: 11) LB (LGR recipe); 12) LB+1% glycerol (LGR recipe); 13) LB+2% glycerol (LGR recipe); 14) LB+3% glycerol (LGR recipe); 15) LB+4% glycerol (LGR recipe); 16) LB+5% glycerol (LGR recipe); 17) LB (Teknova); 18) LB+1% glycerol (derived from Teknova LB+7.5% glycerol); 19) LB+2% glycerol (derived from Teknova LB+7.5% glycerol); 20) LB+3% glycerol (derived from Teknova LB+7.5% glycerol); 21) LB+4% glycerol (derived from Teknova LB+7.5% glycerol); 22) LB+5% glycerol (derived from Teknova LB+7.5% glycerol).

E. coli was transformed and plated onto an LB agar plate and incubated at 37 C for 16 h in a static incubator. Colonies were picked into Echo PP Plus Plates pre-filled with 40 μL per well of each media formulation. The PP Plus Plates were incubated for 24 h at 37 C, 800 rpm and full copy transfers onto an LB agar carbenicillin plate of 25 nL per well performed on the Echo 525. LB agar carbenicillin plates were incubated for 14 h at 30 C in a static incubator.

Of these media formulations, #13-16, #22 were observed to produce the largest colony size (e.g. highest #E. coli transferred). We decided to pursue LB+2.5% glycerol and LB+5% glycerol media formulations for further testing.

To test rates of cross-contamination using these media formulations, Echo PP Plus Plates were pre-filled with 40 μL of the following 5 media formulations: LB, LB+2.5% glycerol (LGR), LB+5% glycerol (LGR), LB+2.5% glycerol (derived from Teknova LB+7.5% glycerol), LB+5% glycerol (derived from Teknova LB+7.5% glycerol). E. coli were transformed and plated onto LB agar carbenicillin plates and incubated at 37 C for 16 h in a static incubator. To stress test rates of cross-contamination, colonies were repeatedly picked into a “wide checkerboard” pattern into the same plate 6 times (576 total picks per plate). Echo PP Plus Plates were then incubated for 48 h at 37 C, 800 rpm in a shaking incubator. The following observations were made: LB: no contamination observed, E. coli pelleting observed; LB+2.5% glycerol (LGR): no contamination observed, minimal E. coli pelleting observed; LB+5% glycerol (LGR): 4 contaminations observed, minimal E. coli pelleting observed; LB+2.5% glycerol (Teknova): 5 contaminations observed, minimal E. coli pelleting observed; LB+5% glycerol (Teknova): 5 contaminations observed, minimal E. coli pelleting observed.

Based on these observations, we selected media formulation LB+2.5% glycerol (LGR) as our custom media formulation for colony picking. The LGR LB+2.5% glycerol recipe is: 15 g tryptone (RPI #T60060), 7.5 g yeast extract (RPI #20020), 7.5 g NaCl (Sigma #S3014), 37.5 mL glycerol (Promega #H5433) and 1462.5 mL DI H2O to produce 1.5 L of media. Once mixed, media is autoclaved and 100 g/mL carbenicillin added prior to use.

### Combinatorial Pooling on Echo 525 and Hamilton STAR

Echo PP Plates (Beckman Coulter #PP-0200) were pre-filled with 40 μL LB and incubated for 72 h at 37 C, 250 rpm. After incubation, PP Plates were allowed to come to RT for 1 h. Combinatorial pooling using 24-bitcodes was performed on the Echo 525 using BioRad 384-well PCR Plates (Bio-Rad #HSP3905) as the destination plate(s), with a maximum fill volume of 12 μl per well and Columns 1-24 representing Pools 1-24. The rows of each column of the destination plate(s) were combined on the Hamilton STAR to produce 24 final pools.

In a ‘first’ experiment, a target library of 2442 guides was picked (6569 colonies picked) at 2.7× coverage and combinatorially pooled using a 24-bitcode scheme. The guide prediction rate was 72% (1754 guides recovered). Based on simulations of the theoretical guide prediction rate as a function of library skew and pick-coverage (FIG. 7B), we identified potential for improvements.

We hypothesized that guide prediction rates could be improved by improving the efficiency of E. coli transfer on the Echo 525. As the Echo 525 uses acoustic-based liquid transfers, we hypothesized that previously observed pelleting of E. coli cultured in LB media could adversely affect E. coli transfer, decreasing guide prediction rates. Alternative media formulations for E. coli colony picking and combinatorial pooling were designed and tested. Based on results detailed in Automated Colony Picking, we identified LB+2.5% glycerol (LGR) as an alternative media formulation that improved E. coli transfer efficiency without generating cross-contamination during the colony picking step. Using this media formulation, we further investigated transfer settings on the Echo 525, testing the “Plus Plate BP” and “Plus Plate GP” settings. The “Plus Plate BP” settings were observed to produce marginally less efficient transfers, but less cross-contamination in the source plate. The “Plus Plate BP” transfer setting was selected for experimental use.

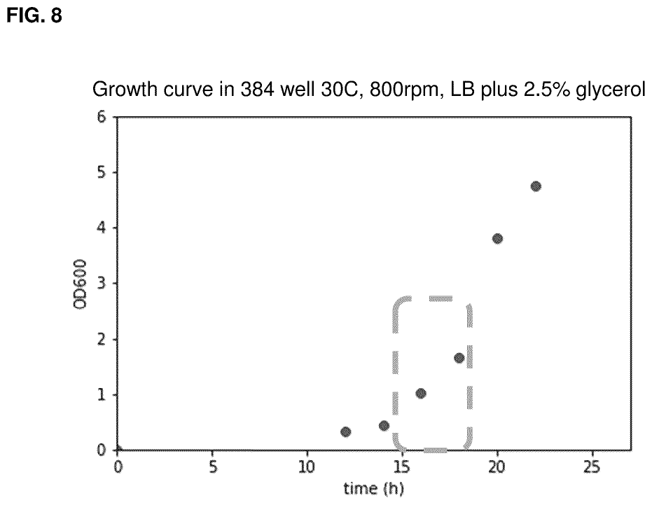

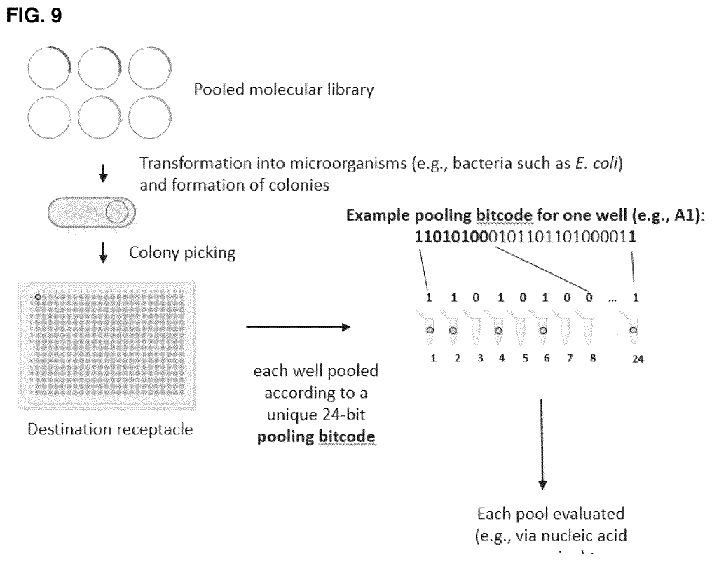

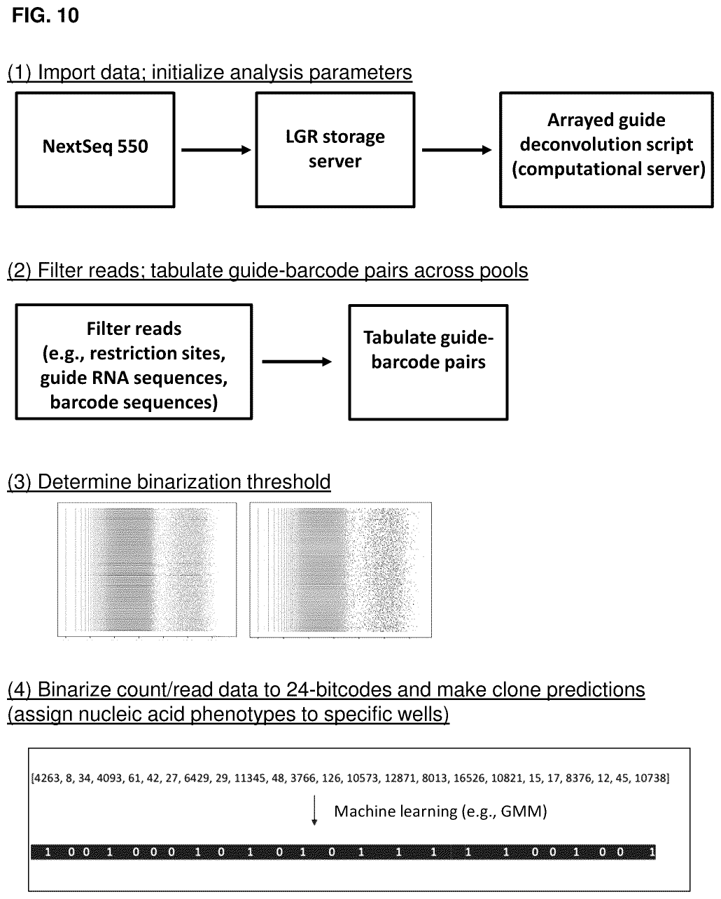

When we used the “Plus Plate BP” transfer setting for combinatorial pooling, we observed cross-contamination in the source plate. These plates were not cross-contaminated after the colony picking step, indicating that cross-contamination was occurring during combinatorial pooling. Based on information from a Labcyte Field Application Scientist, we investigated the hypothesis that cultures in early-to-mid log phase transfer more cleanly than those grown to late-log or stationary phase. We mapped the growth curve for colonies picked into Echo PP Plus Plates pre-filled with 40 μL LB+2.5% glycerol (LGR) and incubated at 37 C, 800 rpm and determined that cultures reached early-to-mid log phase after 10-12 h of incubation. This duration of incubation requires placing the plates in the incubator very late in the evening and/or very early in the morning. To adapt our protocol to standard working hours, we mapped the growth curve for colonies picked into 40 μL LB+2.5% glycerol (LGR) and incubated at 30 C, 800 rpm and determined that cultures reached early-to-mid log phase after 16-18 h of incubation (FIG. 8). We selected this culture condition for experimental use.

Using 40 μL LB+2.5% glycerol (LGR) cultures incubated at 30 C, 800 rpm for 16 h and transferred using the “Plus Plate BP” setting, we continued to observe cross-contamination in the source plate after combinatorial pooling. During these tests, we observed that full copies of the source plate into 384-well Bio-Rad PCR destination plates of volumes ranging from 25 nL to 10,000 nL could be performed cleanly. In our original experimental design, the source plate for combinatorial pooling also served as the source plate for cherry picking. However, as we were unable to achieve contamination-free combinatorial pooling, we hypothesized that we could instead alter our experimental design to produce a copy of each source plate prior to combinatorial pooling. This copy plate would then serve as the source plate during cherry picking.

To determine how to cleanly make copies that would be viable for cherry picking, we tested a range of transfer volumes and backfill settings. We picked E. coli colonies into an Echo PP Plus Plate pre-filled with 40 uL LB+2.5% glycerol (LGR) and incubated the plate at 30 C, 800 rpm for 16 h. We allowed the plates to come to RT for 1 h. We then made full copy transfers using 25 nL/well, 100 nL/well, and 500 nL/well transfers, using Echo PP Plus Plates as the destination plates. We then backfilled the destination plates on the Combi nL with 40 uL LB+2.5% glycerol (LGR) using the Water (Speed 5) setting. We observed no cross-contamination in the 25 nL and 100 nL plates, but did observe cross-contamination in the 500 nL plate. To investigate this further, we made two full copy transfers of 500 nL and back-filled one on the Combi nL and the other manually with a multi-channel pipette. We observed cross-contamination in the destination plate back-filled on the Combi, but no cross-contamination in the destination plate back-filled manually. We hypothesized that the larger the copy volume, the higher the probability of cross-contamination due to splashing on the Combi nL. We decided to produce our copies using 100 nL transfers on the Echo 525 followed by backfilling with 40 uL LB+2.5% glycerol (LGR) on the Combi nL, using Water (Speed 5) liquid dispense settings.

Using this new experimental design, Echo PP Plus Plates (Beckman Coulter #PPL-0200) were pre-filled with 40 μL LB+2.5% glycerol (LGR) and incubated for 16 h at 30 C, 800 rpm. After incubation, PP Plus Plates were allowed to come to RT for 1 h. Copies of each PP Plus Plate were generated. Each PP Plus Plate was then combinatorially pooled on the Echo 525 using 24-bitcodes using BioRad 384-well PCR Plates (Bio-Rad #HSP3905) as the destination plate(s), with a maximum fill volume of 12 μl per well and Columns 1-24 representing Pools 1-24. The rows of each column of the destination plate(s) were combined on the Hamilton STAR to produce 24 final pools.

In a ‘second’ experiment, a target library of 801 guides was picked at 3.6× coverage (2861 colonies picked) and combinatorially pooled using this approach. The guide prediction rate was 90% (717 guides recovered).

Based on a simulation of the theoretical guide prediction rate as a function of the pick coverage and library skew, the prediction rate accomplished in the ‘second’ experiment approximated the theoretical limit (FIG. 7B).

In later tests, we compared the Water (Speed 5), Water (Speed 1), and 30% Glycerol (Speed 5) liquid dispense settings on the Combi nL and determined that 30% Glycerol (Speed 5) produced the lowest rates of cross-contamination. In our current experimental design, we produce copies using 100 nL transfers on the Echo 525 followed by backfilling using 40 uL LB+2.5% glycerol (LGR) on the Combi nL, using Glycerol 30% (Speed 5) liquid dispense settings.

### Next Generation Sequencing Library Preparation

After combinatorial pooling on the Echo 525 and Hamilton STAR, the 24 final pools were miniprepped and quantified. The PS1/PS2/barcode region of each plasmid was then PCR amplified using 24 unique F-primers containing 24 unique i7 indexes, and a universal R-primer. PCR products were quantified and normalized to produce a master pool containing the 24 final pools. One-sided magnetic bead size selection was used to remove excess primer and produce the final sequencing library.

### Next Generation Sequencing

Libraries were sequenced on the NextSeq550 using a High Output 150 Cycle Kit. Custom R1, R2, and i5 sequencing primers were used to sequence PS1, PS2, and the 11 bp barcode, respectively. PhiX was added to the sequencing library to increase base diversity.

### Computational Deconvolution and Guide Prediction

A Python script was written to analyze sequencing data, make guide predictions, and generate a cherry pick list for input into the Echo 525. The script utilizes a machine-learning model to binarize guide counts data and generate a list of guide predictions. Several iterative improvements were made to the script to improve ease of use, run time, and guide prediction rates.

The first step of the script imports FASTQ data produced by the NextSeq 550 and populates a “guide_dict” variable. FASTQ data was filtered for those containing expected PS1_PS2 sequences based on our target library and expected barcode sequences based on our input list of 42 barcodes. The “guide_dict” variable is a dictionary whose keys are unique guide1_guide2_barcode sequences and whose values are 24-element arrays containing the number of observed reads in each of 24 pools that correspond to each key. An example entry is:

GCTACGCCCGGGGGAAAAGA_GCAGGAGCTAAGGGTCCCGT_ACCAGCCGATG:
(SEQ ID NO: xx)
[1, 3, 37305, 0, 5, 26502, 20, 56173, 6, 3, 41560, 39019, 2,
42514, 12, 5, 29151, 61658, 38040, 31852, 29176, 16, 21, 19468]

This entry indicates that this PS1_PS2_barcode sequence was observed 1 time in Pool 1, 3 times in Pool 2, 37305 times in Pool 3, etc.

With our sequencing approach, FASTQ data is generated by the NextSeq 550 in 24 different files corresponding to the 24 different indexes used to PCR amplify the 24 pools. Initially, these FASTQ files were processed sequentially, resulting in a run time of several hours to populate the “guide_dict” variable. To improve run time, we parallelized population of each column of the “guide_dict” variable over 24 different computational cores, combining these data into 24-element arrays once all 24 pools had been processed. This parallelization reduced the run time for populating the “guide_dict” variable from several hours to a few minutes.

Once the “guide_dict” variable was populated, we plotted the raw counts data as a scatter plot and manually identified the binarization threshold. “High” counts translated to a “1” in the pooling bitcode, while “low” counts translated to a “0” in the pooling bitcode. Identification of the binarization threshold was not automated and thus had to be manually determined for each run. Additionally, manual identification of the binarization threshold is subject to user bias or error. Additionally, as we increased the scale of arrayed libraries we were attempting to generate, each clone represented an increasingly smaller fraction of the overall number of reads, effectively lowering the signal-to-noise ratio and increasing the difficulty of manual threshold identification.

In addition to difficulties in manual threshold identification, we observed variability in the total number of sequences identified in Pool 1 vs. Pool 2 vs. Pool 3, etc. (e.g. variability in the sum of the 0^{th} element in the arrays vs. the 1^{st} element vs the 2^{nd} element, etc.). Experimentally, the sequencing library is made by quantifying the PCR product resulting from each of the 24 pools and normalizing these into a single pool. This approach decreased, but did not eliminate, variability in the representation of each pool in the library. In order to determine a global binarization threshold across all 24 pools, we normalized the counts data for each value in the dictionary by multiplying each count by the maximum sum counts of all pools divided by the sum counts for the pool to which that count belonged. These data were used to populate a “guide_dict_normalized” dictionary. An example entry is:

GCTACGCCCGGGGGAAAAGA_GCAGGAGCTAAGGGTCCCGT_ACCAGCCGATG:
(SEQ ID NO: xx)
[2, 3, 45670, 0, 6, 33809, 26, 60716, 8, 3, 51089, 67031, 3,
47698, 14, 5, 39957, 61658, 45866, 39765, 35584, 23, 29, 53400]

Comparing the value for the same PS1_PS2_barcode sequence in the “guide_dict_normalized” variable versus that in the “guide_dict” variable, we observe linear scaling of all counts data in the array other than the 18^{th} count, which corresponds to the pool with the maximum number of counts.

Once the “guide_dict_normalized” variable had been populated, we began training ML models for automated threshold determination and binarization of the counts data. As we did this, we observed that some of the keys in the “guide_dict_normalized” variable likely corresponded to true guides that merited downstream analysis, while others likely corresponded to spurious guides. Example entries are below:

### Hypothesized True Guide:

GCTACGCCCGGGGGAAAAGA_GCAGGAGCTAAGGGTCCCGT_ACCAGCCGATG:
(SEQ ID NO: xx)
[2, 3, 45670, 0, 6, 33809, 26, 60716, 8, 3, 51089, 67031, 3,
47698, 14, 5, 39957, 61658, 45866, 39765, 35584, 23, 29, 53400]

### Hypothesized Spurious Guide:

GGGAGGTACCGGCTGTTGTG GGGCAGAGCCGCACAACAGC_AGATTCCGCC:
(SEQ ID NO: xx)
[0, 0, 0, 1, 0, 3, 0, 0, 0, 0, 0, 0, 0, 0, 0, 0, 0, 0, 0, 0,
0, 0, 0, 3]

To prevent data from spurious guides from affecting training of our ML model for counts binarization, we trained a different ML model to differentiate true guides from spurious guides. We reasoned that the sum of counts across each 24-element array would be much higher for true guides than for spurious guides. For each key in the “guide_dict_normalized” variable, we summed across each count in the 24-element array. For the above two guides, these values were 582365 and 7.

We then trained a k-means clustering algorithm (k=2) on this dataset, but were not able to achieve robust threshold identification. We hypothesized that this was due to high variance in the dataset that led to difficulties in distinguishing true guides with low sum counts from spurious guides. To address this issue, we log-transformed the sum counts data and trained a k-means clustering algorithm (k=2) on this dataset. This improved the model's predictive ability, but threshold identification did not always align with that determined by the user. We hypothesized that this was due to parameter constraints enforced by the k-means clustering algorithm. K-means clustering algorithms enforce that all clusters share identical radii (e.g. all clusters are of the same size and shape). We hypothesized that these constraints were inappropriate for our dataset and enforcement of the “signal” cluster being the same size as the “noise” cluster could adversely affect model learning and prediction. To address these issues, we trained a 2D Gaussian Mixture Model (GMM, n=2) on the log-transformed dataset, with each component having its own general covariance matrix. This allowed for the model to predict clusters of dissimilar size as well as elliptical, rather than circular, clusters.

Using this approach, we were able to achieve robust, automated threshold identification using a GMM to differentiate between true guides and spurious guides. By applying this model to discard spurious guides, we were able to populate a “guide_dict_reduced” dictionary, which contains only keys believed to correspond to true guides.

Once the “guide_dict_reduced” variable was populated, we trained an ML model to distinguish “high” counts from “low” counts for individual PS1_PS2_barcode sequences. Our attempt to develop an ML model for this mirrored our attempts to develop a ML model for distinguishing true guides from spurious guides. When we attempted to train a k-means clustering algorithm (k=2) on counts data in the “guide_dict_reduced” variable, we were unable to achieve robust threshold identification. This was hypothesized to be due to high variance in the data that made it difficult to distinguish low “high” counts from true “low” counts. To address this issue, we log-transformed the data and used the transformed counts to train a k-means clustering algorithm (k=2). Using this approach, we observed unreliable threshold identification that did not always align with that determined by the user. To address these issues, we trained a 2D Gaussian Mixture Model (GMM, n=2) on the log-transformed dataset, with each component having its own general covariance matrix. This allowed for the model to predict clusters of dissimilar size as well as elliptical, rather than circular, clusters. Using the GMM model, we achieved robust, automated identification of binarization thresholds.

We then applied this GMM to values in the “guide_dict_reduced” variable, converting “high” counts data to “1” and “low” counts data to “0”. This data was then used to populate a “guide_dict_binary” variable, where each key is a unique PS1_PS2_barcode sequence, and each value is the GMM-binarized counts data for that key. An example entry is:

GCTACGCCCGGGGGAAAAGA_GCAGGAGCTAAGGGTCCCGT_
ACCAGCCGATG:
001001010011010011111001

The values in the “guide_dict_binary” variable (termed “experimental bitcodes”) were then matched against “pooling bitcodes” that were used on the Echo 525 for combinatorial pooling (Combinatorial Pooling on Echo 525 and Hamilton STAR), with matches written to file. Entries in this file consisted of a PS1_PS2_barcode sequence and its predicted plate and well location. An example entry is:

GGAGCCGCGGGCGGTCAGGT_GGCTCCGACGAGTCCACCGC_
CGCGCACAGTT 1 A3

This entry indicates that the clone containing this PS1_PS2_barcode sequence is predicted to reside in Plate 1, Well A3. All “experimental bitcodes” for which there was a matched “pooling bitcode” resulted in a prediction in this file.

Before using the guide prediction file for cherry-picking, we culled two undesirable types of guide predictions. Firstly, we removed predictions in which two or more unique PS1_PS2_barcode sequences were predicted to reside in the same plate and well. An example of this is:

GACCATGGTAGCGATGTCAG_GACATCGCTACCATGGTCTC_
TCCGAGATGGG:
1O22
GCTGGGCAGCGGAAGAAGGG_GCAGCCCCGGAACGCCATCG_
GCCGACCACTC:
1O22

We hypothesized that these mixed predictions resulted from more than one plasmid being transformed into a single E. coli, producing a hybrid clone, or from more than one colony being picked during automated colony picking. Mixed clones are undesirable in an arrayed library and were not included in the list of guides for cherry picking.

Secondly, we removed redundant PS1_PS2 sequences from the list of guide predictions. An example of this is:

GGAGCCGCGGGCGGTCAGGT_GGCTCCGACGAGTCCACCGC_
CGCGCACAGTT:
1A3
GGAGCCGCGGGCGGTCAGGT_GGCTCCGACGAGTCCACCGC_
TGACCGATGCC:
1A23

These predictions share the same PS1_PS2 sequences, but different barcode sequences. The different barcode sequences aided in deconvolution and guide prediction; however, in our arrayed library we only want to maintain one copy of each PS1_PS2 sequence, as this represents the functional guide region of the vector. To address this issue, we only include one instance of each unique PS1_PS2 sequence in the list of guides for cherry picking.

Once the undesirable guide predictions had been culled, the script generated an Echo pick list representing instructions for transferring cultures from a designated plate and well of the copy plate(s), to designated wells of the destination plate(s). An example entry is:
AllClones_1 A3 CherryPick_1 A1 100

This entry instructs the Echo to transfer 100 nL of sample from “A3” of “AllClones_1” (copy plate) to “A1” of “CherryPick_1” (destination plate).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of creating an arrayed library of cells, comprising:
a) performing automated colony selection, comprising:
i) selecting colonies of cells from a colony source and depositing each of the colonies into a unique location of a first receptacle using an automated apparatus for automatic handling of one or more pick tools, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle; and
b) pooling the cells from the selected colonies, comprising:
i) acoustically transferring, using an acoustic liquid handler, a portion of the cells from each said unique location of the first receptacle, wherein the first receptacle contains a high-density media, to one or more locations of a second receptacle to produce cell pools;
c) sequencing nucleic acids of the cell pools to obtain nucleotide sequences; and
d) deconvoluting the obtained nucleotide sequences to assign a nucleic acid genotype to each unique location of the first receptacle.

2. The method of claim 1, wherein the cells are genetically modified.

3. The method of claim 2, wherein the genetically modified cells comprise members of a molecular library.

4. The method of claim 3, wherein the molecular library comprises a plurality of nucleic acids comprising one or more nucleic acid barcode sequences.

5. The method of claim 3, wherein the molecular library is a guide RNA library, a transgene library, an shRNA library, a long noncoding RNA library, an open reading frame expression library, a library of mutated nucleic acid sequences, a library of viral nucleic acid sequences, or any combination thereof.

6-9. (canceled)

10. The method of claim 1, wherein the cells are bacteria, parasites, or fungi.

11-14. (canceled)

15. The method of claim 1, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.

16. The method of claim 1, wherein the first receptacle is a multi-well plate suitable for use with an acoustic liquid handler.

17. The method of claim 1, wherein the high-density media comprises LB broth and 1-10% glycerol.

18-19. (canceled)

20. The method of claim 1, wherein the one or more locations of the second receptacle into which the cells are transferred from the first receptacle are determined using a bitcode sample pooling scheme.

21-22. (canceled)

23. The method of claim 1, wherein the acoustic transfer occurs when the cells are in early- to mid-log phase.

24. The method of claim 1, wherein deconvoluting the obtained nucleic acid sequences comprises using an unsupervised machine learning algorithm.

25. (canceled)

26. The method of claim 1, further comprising employing the nucleic acid genotype and associated unique location of the first receptacle to select and transfer cells from the first receptacle to a third receptable, thus producing a curated arrayed library of cells.

27. The method of claim 1, further comprising preparing a copy of the first receptacle following automated colony selection and prior to pooling the cells.

28-29. (canceled)

30. A system for selecting and transferring cells, comprising:
a) an automated apparatus for automatic handling of one or more pick tools and configured for performing automated selection of colonies of cells into one or more locations of a first receptacle, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle;
b) the first receptacle, comprising a high-density media;
c) an acoustic liquid handler configured to acoustically transfer a portion of the cells from the first receptacle to one or more locations of a second receptacle.

31. (canceled)

32. The system of claim 30, further comprising cells, wherein the cells are genetically modified and comprise members of a molecular library.

33-34. (canceled)

35. The system of claim 30, wherein the automated apparatus for automatic handling of one or more pick tools comprises a modification to reduce the speed at which the active pick tool is extended and retracted during colony selection.

36. (canceled)

37. The system of claim 30, wherein the first receptacle is a multi-well plate suitable for use with an acoustic liquid handler.

38. The system of claim 30, wherein the high-density media comprises LB broth and 1-10% glycerol.

39-41. (canceled)

42. The system of claim 30, further comprising a computer configured for deconvolution of nucleic acid sequences.

43-44. (canceled)

45. A method of selecting and transferring cells, comprising:
a) performing automated colony selection, comprising:
i) selecting colonies of cells from a colony source and depositing the colonies in one or more locations of a first receptacle using an automated apparatus for automatic handling of one or more pick tools, wherein the automated apparatus comprises a solid barrier comprising an opening for an active pick tool to access the colony source and the first receptacle, and wherein the solid barrier is mounted on the automated apparatus and configured to prevent transfer of cells from an inactive pick tool into an undesired location of the first receptacle; and
b) acoustically transferring, using an acoustic liquid handler, a portion of cells from the first receptacle, wherein the first receptacle contains a high-density media, to one or more locations of a second receptacle.

46. (canceled)

47. The method of claim 45, wherein the automated apparatus for automatic handling of one or more pick tools is modified to reduce the speed at which the active pick tool is extended and retracted during colony selection.

48. (canceled)

49. The method of claim 45, wherein the high-density media is added to the first receptacle prior to performing automated colony selection.

50-52. (canceled)

53. A method of acoustically transferring cells, comprising:
a) obtaining a first receptacle comprising one or more cells in a high-density media; and
b) acoustically transferring, using an acoustic liquid handler, at least one of said one or more cells in the high-density media from the first receptacle to one or more locations of a second receptacle.

54. The method of claim 53, wherein the high-density media comprises about 2.5% glycerol.

55-56. (canceled)

57. A system for acoustically transferring cells, comprising:
a) one or more cells in a first receptacle comprising a high-density media;
b) an acoustic liquid handler configured to acoustically transfer at least one of said one or more cells from the first receptacle to one or more locations of a second receptacle.

58. The system of claim 57, wherein the high-density media comprises about 2.5% glycerol.

59-60. (canceled)

61. A method for reducing contamination during automated colony selection, comprising selecting colonies of cells from a colony source and depositing the colonies in one or more locations of a destination receptacle using an automated apparatus for automatic handling of one or more pick tools, wherein the automated apparatus comprises a solid barrier comprising an opening for an active pick tool to access the colony source and the destination receptacle, and wherein the solid barrier is mounted on the automated apparatus and configured to prevent transfer of cells from an inactive pick tool into an undesired location of the destination receptacle.

62-64. (canceled)

65. A solid barrier mounted on an automated apparatus for automatic handling of one or more pick tools, wherein
a) the pick tools are configured to select colonies of cells from a colony source and deposit the colonies in one or more locations of a destination receptacle;
b) the solid barrier comprises an opening for an active pick tool to access the colony source and the destination receptacle; and
c) the solid barrier is configured to prevent transfer of cells from an inactive pick tool into an undesired location of a destination receptacle.

66. The solid barrier of claim 65, wherein the solid barrier comprises acrylic, polycarbonate, polymethyl methacrylate, polyvinyl chloride (PVC), polyethylene terephthalate (PET), polystyrene, polypropylene, acrylonitrile butadiene styrene (ABS), polyethylene, polyethylene terephthalate glycol (PETG), perfluoroalkoxy alkane (PFA), polytetrafluoroethylene (PTFE), polyurethane, fiberglass, glass, ceramic, metal, or any combination of materials thereof.

67. The solid barrier of claim 65, wherein the automated apparatus for automatic handling of one or more pick tools is a Hudson Robotic RapidPick MP.

68. (canceled)
